(19) United States
(12) Patent Application Publication
(10) Pub. No.: US 2007/0265216 A1
(43) Pub. Date: Nov. 15, 2007

(54) ENHANCED MEDICAL TREATMENT IN DIABETIC CARDIOMYOPATHY

(76) Inventors: Richard W. Gross, Chesterfield, MO (US); Xianlin Han, Clayton, MO (US)

Correspondence Address: PATRICK W. RASCHE (15060);ARMSTRONG TEASDALE, LLP, ONE METROPOLITAN SQUARE, SUITE 2600, SAINT LOUIS, MO, 63102-2740, US

(21) Appl. No.: 11/539,136

(22) Filed: Oct. 5, 2006

Related U.S. Application Data
(60) Provisional application No. 60/724,116, filed on Oct. 5, 2005.

(51) Int. Cl. A61K 31/711; A61P 3/10; C12Q 1/00

(52) U.S. Cl. 514/44; 435/4

(57) ABSTRACT

A method of treating a living mammal having diabetic cardiomyopathy comprises administering an effective amount of an inhibitor to the mammal performing a shotgun lipidomics analysis on the mammal and determining that the treatment was successful when and if serum or tissue biopsy cardiolipin levels are increased and/or lysocardiolipin levels are decreased.

### CROSS-REFERENCE TO RELATED APPLICATIONS

This application claims the priority of U.S. Provisional Patent Application Ser. No. 60/724,116 filed Oct. 5, 2005, which is hereby incorporated by referenced in its entirety.

### STATEMENT REGARDING FEDERALLY SPONSORED RESEARCH AND DEVELOPMENT

This invention was made under NIH contracts 5PO1H657278 & 5R01H241250. The government has certain rights in the invention.

### BACKGROUND OF THE INVENTION

This invention relates generally to diabetic marker screening and more particularly to identifying new targets for pharmacologic inhibition. This invention relates generally to analytical (assays) methods for identifying compounds useful for promoting health in living mammalian systems. In particular this invention relates to assays and analytical tools for monitoring health in living mammals.

Diabetic cardiomyopathy is characterized chemically by the presence of marked alterations in the lipid composition of diabetic myocardium, altered substrate utilization and mitochondrial dysfunction (1-9). It is widely believed that mitochondrial dysfunction and inefficient energy production is the underlying cause of hemodynamic alterations in diabetic myocardium. Maladaptive responses to persistent changes in substrate utilization (e.g., increased fatty acid uptake and decreased glucose transport) and metabolic flux lead to the accumulation of toxic lipids in diabetic myocardium (e.g., acyl carnitines, acyl CoA, and triacylglycerols) which are thought to contribute to hemodynamic dysfunction (2, 3, 6, 8, 9). In early studies, we and others, identified profound alterations in the lipid composition in obese and diabetic myocardium from rats which were accompanied by physiologic dysfunction (1, 2). Further work clarified the importance of alterations in substrate utilization, in particular, in the increased reliance on fatty acid substrate, as an important contributor to the diastolic dysfunction present in diabetic myocardium (1, 8). However, the molecular mechanisms through which altered myocardial fatty acid and glucose substrate utilization and mitochondrial dysfunction are chemically linked in the diabetic state are not understood.

Historically, inborn errors of metabolism have provided fundamental insights into the sequence of chemical events underlying a multitude of both physiological and pathophysiological processes. Traditionally, inherited disorders of metabolism have been identified through phenotypic alterations (signs, symptoms, pathology, and laboratory assessments) and the responsible chemical mechanisms have been deduced from biochemical, physiological, or genetic approaches. Recently, the development of lipidomics using mass spectrometry has provided a detailed metabolic fingerprint which can be utilized to compare alterations in the disease state of interest with those manifest in a growing body of known genetic diseases and nutritional disorders (10-18).

Through comparisons of the similarities and differences of alterations in lipid profiles with known genetic diseases, in conjunction with increasing knowledge of the roles of specific lipids in physiological and pathophysiological processes, significant insight into the chemical mechanisms underlying complex disease processes can be accrued (9, 19-23). One such genetic abnormality, Barth's syndrome, results from mutations in the X chromosome located at Xq28 that is characterized clinically by neutropenia, skeletal myopathy, and cardiomyopathy. This disorder has been traced to alterations in cardiolipin metabolism (24-28) resulting in cardiolipin depletion, mitochondrial dysfunction, and cardiomyopathy.

During the last decade, excessive consumption of fat in high caloric Western diets in conjunction with a sedentary life style, has resulted in an epidemic of obesity in industrialized nations (1, 2). Obesity is associated with insulin resistance, hypertension, dyslipidemia, type 2 diabetes and atherosclerosis, which collectively constitute the metabolic syndrome (3, 4, 5). Despite the enormous proportions of this public health problem, the biochemical mechanisms underlying the metabolic syndrome and its end-organ sequelae are poorly understood.

With respect to diabetes, glucose utilization is necessary for the body to be able to use sugar which is stored in the blood as glucose. Insulin initiates the process of taking glucose from the blood and moving it into the cells. However, when glucose builds up in the blood instead of going into cells (e.g., insulin resistance), it can cause serious life threatening problems which results in type 2 diabetes. These include heart disease (cardiovascular disease), blindness (retinopathy), nerve damage (neuropathy), and kidney damage (nephropathy).

Type 2 diabetes is the most common form of diabetes. In this condition the body does not produce enough insulin to cause cells to transport glucose or the cells are not sensitive enough to the insulin present. The concentration of blood glucose becomes and remains high in the blood resulting in unnecessary and undesired damage to the body. Thus glucose is not utilized, proteins are covalently modified, inappropriate oxidation occurs and a change to fatty acid substrate occurs. Increases in intracellular lipids occur (lipotoxicity) as well as mitochondrial dysfunction leading to accumulation of toxic metabolites such as acyl-CoAs, acyl-carnitines, and diglycerides. Covalent modification by palmitoylation may occur in excess leading to dysfunctional body metabolism.

While tremendous strides have been made in medical research in this area, it is highly desired to identify a composition which treats diabetes in particular diabetic myopathy. Moreover, it is highly desired to have a method for identifying compounds which are useful to treat diabetic myopathy in living mammalian systems and to use these compounds to improve lifestyle. It is also highly desired to have an effective diagnostic tool to diagnose the present of diabetic myopathy.

Recent studies have underscored the role of lipotoxicity and mitochondrial dysfunction in the pathophysiology of diabetic cardiomyopathy. However, the biochemical mechanisms underlying these alterations are incompletely understood.

New and enhanced methods of treating diabetes are needed along with identification of new targets for pharmacologic inhibition.

### BRIEF DESCRIPTION OF THE DRAWINGS

FIGS. 1A and 1B depict representative negative-ion electrospray ionization mass spectra of myocardial lipid extracts from control and diabetic mice.

FIGS. 2A and 2B depict two-dimensional electrospray ionization mass spectrometric analyses of anionic phospholipids in myocardial chloroform extracts of a control and diabetic mouse.

FIGS. 3A and 3B depict comparison of cardiolipin and phosphati-dylglycerol mass content in lipid extracts of diabetic and control mouse myocardium.

FIGS. 4A and 4B depict comparison of cytochrome c, ATP synthase β, and glycerol-3-phosphate mass content in myocardial homogenates of diabetic and control mice.

FIGS. 5A and 5B depict triacylglycerol molecular species analyses by two dimensional electrospray ionization mass spectrometry of myocardial chloroform extracts from control or diabetic mice.

FIGS. 6A and 6B depict representative negative-ion electrospray ionization mass spectra of myocardial lipid extracts from control and diabetic mice using intrasource separation.

FIGS. 7A and 7B depict two-dimensional electrospray ionization mass spectrometric analyses of myocardial chloroform extracts of control or diabetic mice using intrasource separation.

### DETAILED DESCRIPTION OF THE DRAWINGS

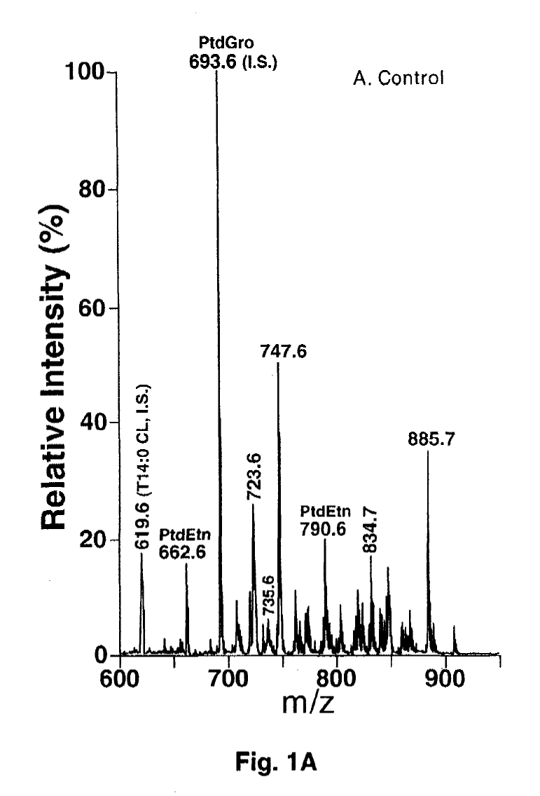

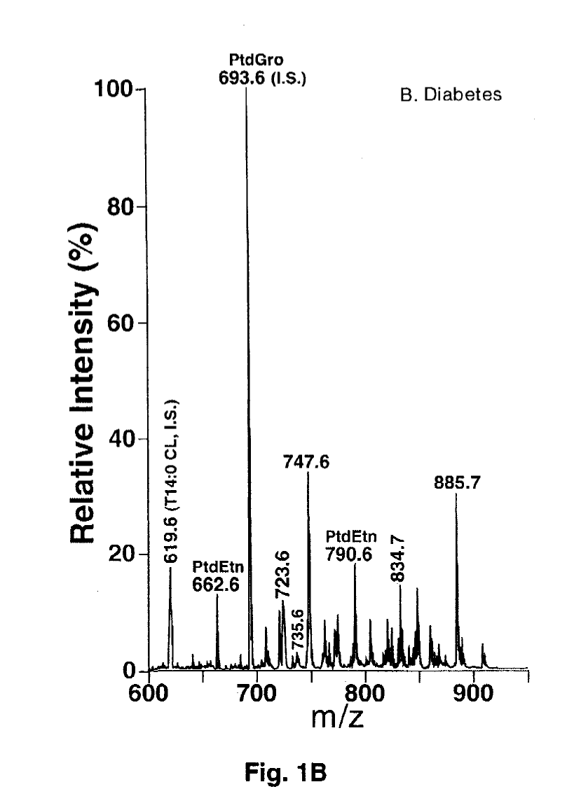

FIGS. 1A and 1B depict representative negative-ion electrospray ionization mass spectra of myocardial lipid extracts from control and diabetic mice. Lipids were extracted from myocardium of a control (Panel A) or diabetic (Panel B) mouse by a modified Bligh and Dyer procedure. Negative-ion ESI/MS of diluted mouse myocardial lipid extracts were performed as described in Materials and Methods. The peaks of the predominant molecular species of cardiolipin (tetra 18:2, m/z 723.6) and phosphatidylglycerol (16:0-18:1, m/z 747.6) were substantially depleted in the diabetic state. Molecular species of other negatively-charged phospholipids including 18:0-20:4 Ptdlns (m/z 885.7) or zwitterionic phospholipids such as 18:0-22:6 PtdEtn (m/z 790.6) were not significantly depleted.

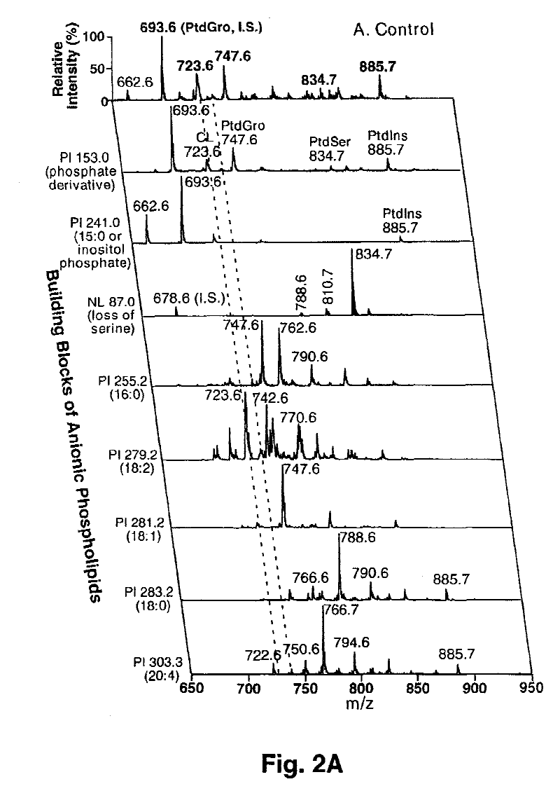

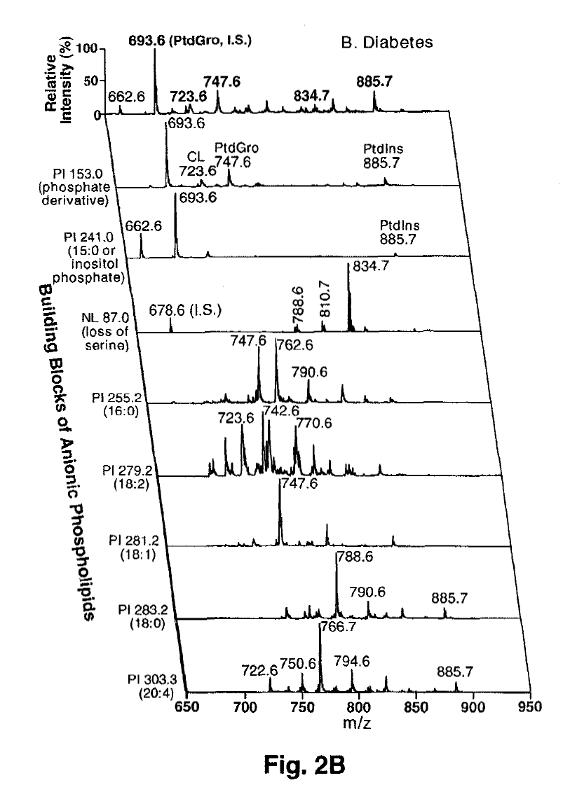

FIGS. 2A and 2B depict two-dimensional electrospray ionization mass spectrometric analyses of anionic phospholipids in myocardial chloroform extracts of a control or diabetic mouse. Identical diluted lipid extracts from a control (Panel A) or diabetic (Panel B) mice were used as described in FIG. 1. Each MS or MS/MS trace of 2D ESI mass spectra was acquired by sequentially programmed custom scans operating under Xcalibur software as described in Materials and Methods. For negative-ion tandem mass spectrometry in the precursor-ion (PT) mode, the first quadrupole was scanned in the selected mass range and the second quadrupole was used as a collision cell while the third quadrupole was fixed to monitor the ion of interest (i.e., either a polar head group of phospholipid or a fatty acyl carboxylate fragmented from anionic phospholipid molecular species). For tandem mass spectrometry in the negative-ion neutral loss (NL) mode, both the first and third quadrupoles were coordinately scanned with a mass difference (i.e., neutral loss) of 87 u, corresponding to the neutral loss of serine from phosphatidylserine molecular species, while collisional activation was performed in the second quadrupole. All mass spectral traces were displayed after normalization to the most intense peak (base peak) in each individual trace. “I.S.” denotes internal standard; “CL” represents doubly-charged cardiolipin. The results demonstrate the predominance of the tetra 18:2 cardiolipin molecular species along with minor amounts of 18:2-18:2-18:2-18:1, 18:2-18:2-18:2-20:4, and 18:0-18:2-18:2-20:4 cardiolipin molecular species at m/z 724.6, 735.6, and 737.6, respectively, as their doubly-charged ions

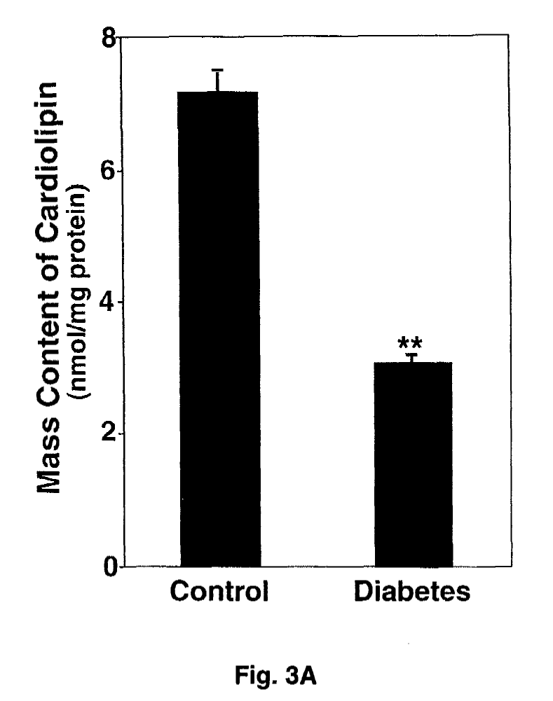

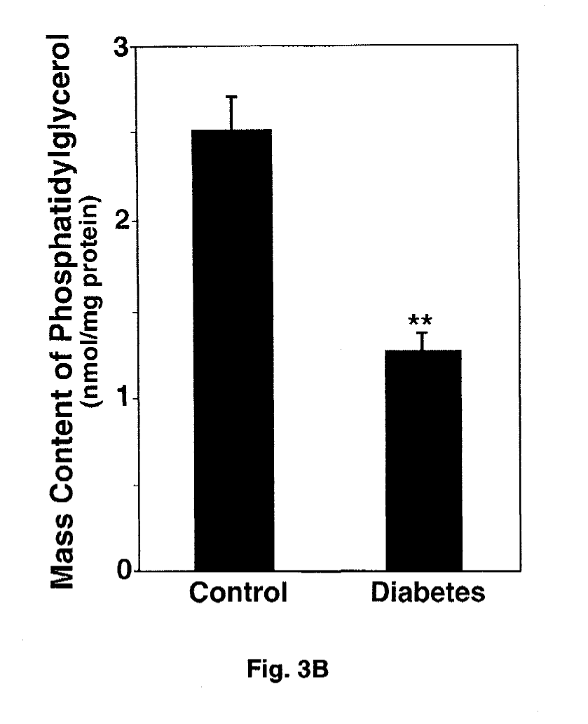

FIGS. 3A and 3B depict comparison of cardiolipin and phosphatidylglycerol mass content in lipid extracts of diabetic and control mouse myocardium. Mass content of cardiolipin (panel A) and phosphatidylglycerol (panel B) was determined by multi-dimensional mass spectrometric array analyses by comparison of the peak intensity of each individual ion to that of the selected internal standards after corrections for ¹³C isotopomer distribution differences as described in the Materials and Methods. **p<0.0001 with n=7.

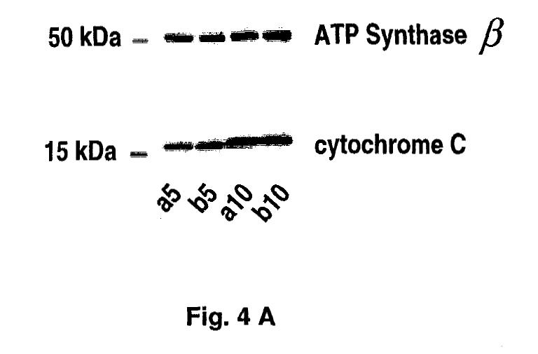

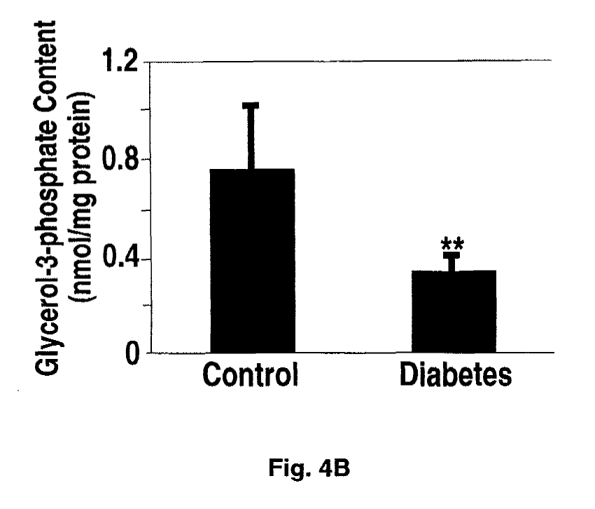

FIGS. 4A and 4B depict comparison of cytochrome c, ATP synthase β, and glycerol-3-phosphate mass content in myocardial homogenates of diabetic and control mice. Panel A: Five (lanes a5 and b5) or ten (lanes a10 and b10) micrograms of protein from control (lanes a5 and a10) or diabetic (lanes b5 and b10) mouse myocardium were loaded onto the gel and electrophoresed by SDS-PAGE. Proteins were transferred to PVDF membranes and subjected to Western blotting with anti-cytochrome c or anti-ATP synthase β antibody as described in Materials and Methods. Panel B: Glycerol 3-phosphate levels in control and diabetic myocardium were determined using a spectrophotometric assay based on the conversion of NADH to NAD in an enzymatic cycling system as described in Materials and Methods. **p<0.01, n=4.

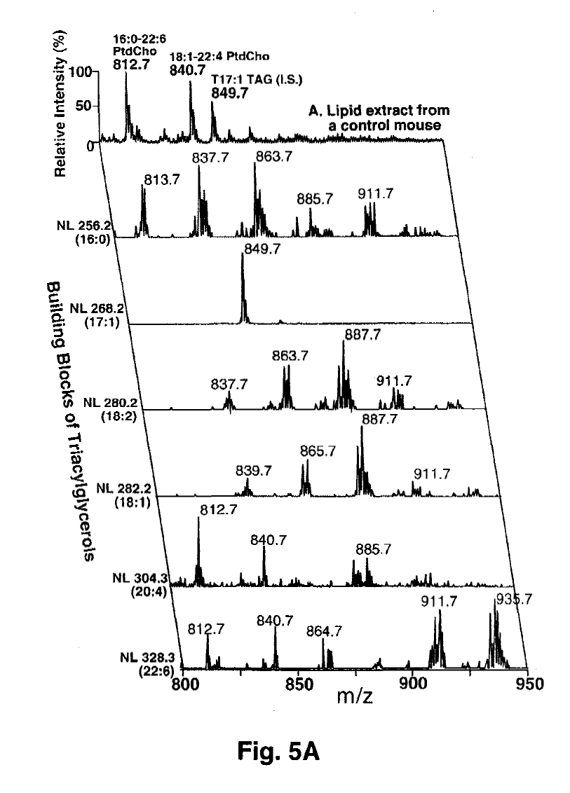

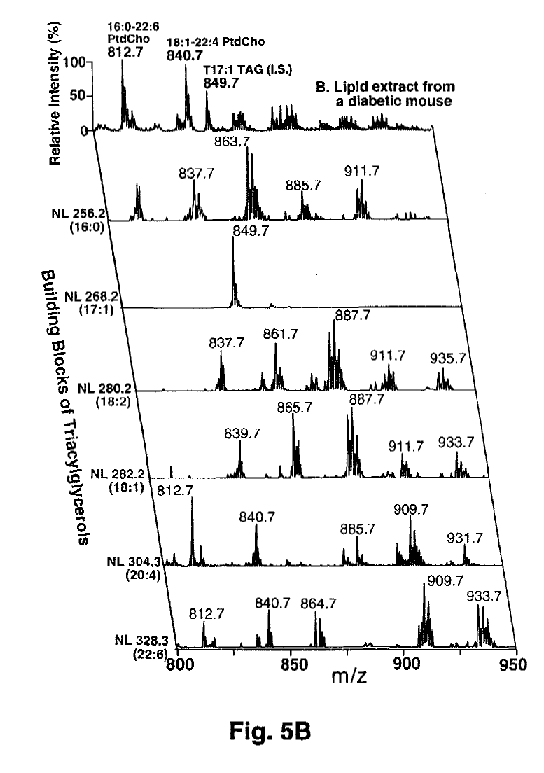

FIGS. 5A and 5B depict triacylglycerol molecular species analyses by two dimensional electrospray ionization mass spectrometry of myocardial chloroform extracts from control or diabetic mice. First dimension spectra were obtained (top trace) in the positive-ion mode using intrasource separation. Next, neutral loss (NL) scanning of all naturally-occurring aliphatic chains (i.e., the building blocks of TAG molecular species) of myocardial chloroform extracts of control (left panel) and diabetic mice (right panel) were utilized to identify the molecular species assignments, deconvolute isobaric molecular species, and quantify triacylglycerol individual molecular species by comparisons with a selected internal standard. The results were remarkable for the altered content and distribution of 20:4-containing TAG molecular species (NL scan at 304.3) in diabetic myocardium demonstrating altered eicosanoid metabolism in the diabetic state. Each MS or MS/MS trace of 2D ESI mass spectra was acquired by sequentially programmed custom scans operating under Xcalibur software as described in Materials and Methods. For tandem mass spectrometry in the positive-ion neutral loss (NL) mode, both the first and third quadrupoles were coordinately scanned with a mass difference (i.e., neutral loss) corresponding to the neutral loss of a non-esterified fatty acid from TAG molecular species, while collisional activation was performed in the second quadrupole. All mass spectral traces were displayed after normalization to the base peak in the individual spectrum.

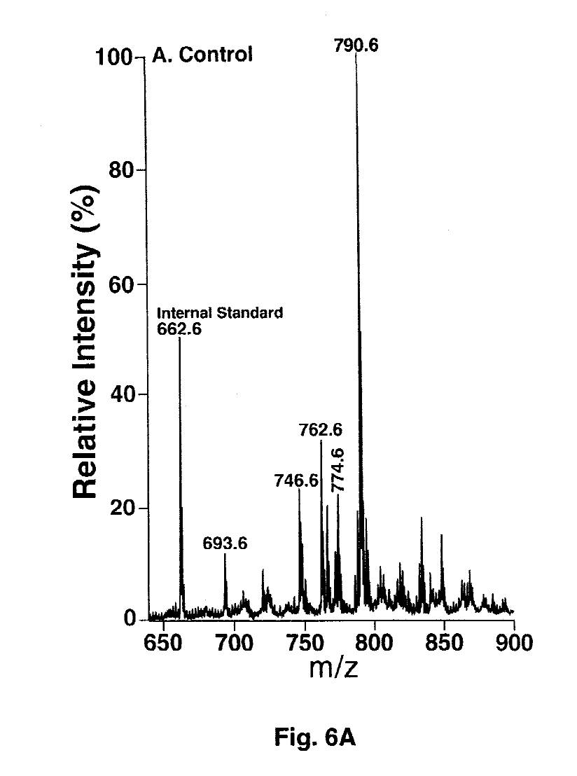

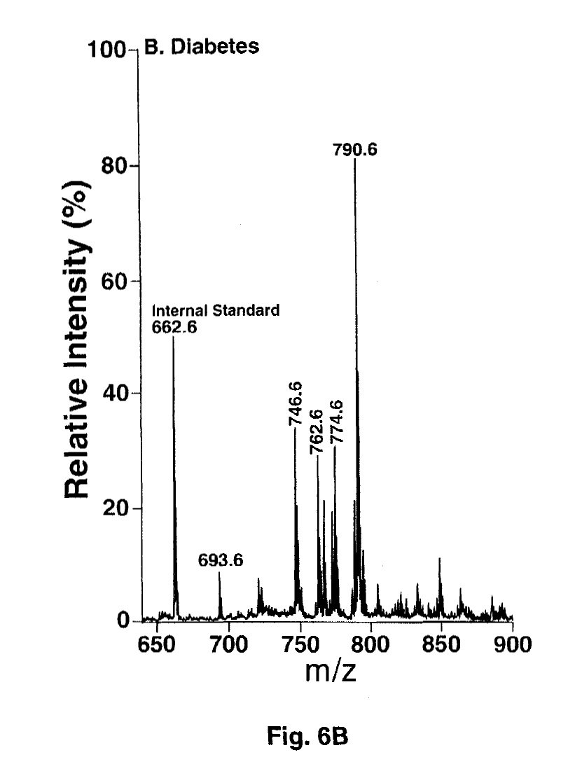

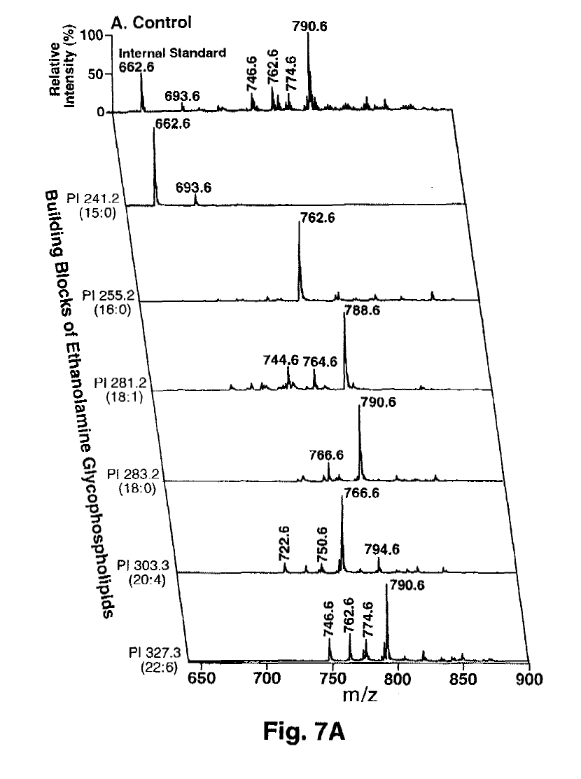

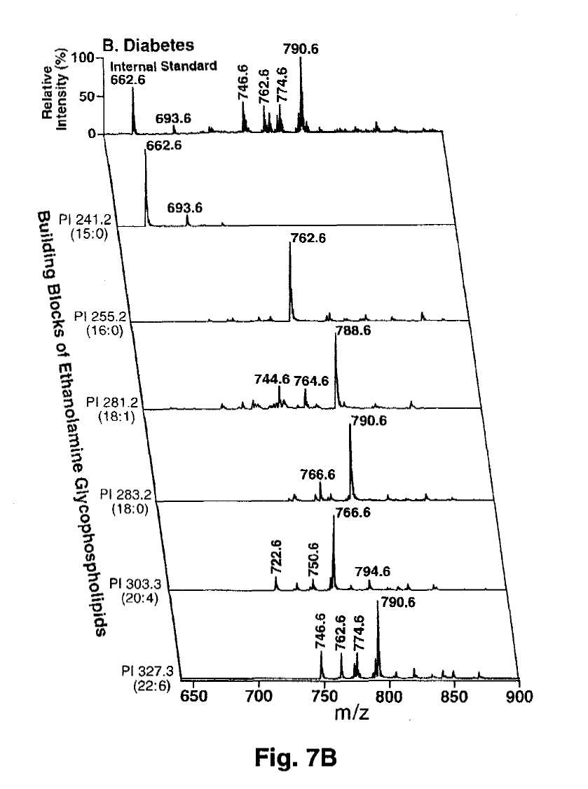

FIGS. 6A and 6B depict representative negative-ion electrospray ionization mass spectra of myocardial lipid extracts from control and diabetic mice using intrasource separation. Lipid extracts from control and diabetic mice were prepared by a modified Bligh and Dyer procedure identical to those described in the legend of FIG. 1. Negative-ion ESI mass spectra of diluted mouse myocardial lipid extracts in the presence of a small amount of LiOH were acquired as described in Materials and Methods. All the abundant molecular ion peaks were identified as PE molecular species by 2D mass spectrometric analyses in FIG. 7. Ion peaks at m/z 746.6, 772.6, and 774.6 are 16:0-22:6, 18:1-22:6, and 18:0-22:6 PlsEtn molecular species, respectively, whereas ion peaks at m/z 762.6 and 790.6 are 16:0-22:6 and 18:0-22:6 PtdEtn molecular species, respectively. The results demonstrate an increase in plasmenylethanolamine subclass and a decrease in phosphatidylethanolamine subclass in ethanolamine glycerophospholipids while the total mass of the ethanolamine glycerophospholipid class is essentially identical in control and diabetic myocardium.

FIGS. 7A and 7B depict two-dimensional electrospray ionization mass spectrometric analyses of myocardial chloroform extracts of control and diabetic mice using intrasource separation. The identical diluted lipid extracts from a control (Panel A) and diabetic (Panel B) mouse used for FIG. 6 were employed. Each MS or MS/MS trace of 2D ESI mass spectra was acquired by sequentially programmed custom scans operating under Xcalibur software as described in Materials and Methods. For negative-ion tandem mass spectrometry in the precursor-ion (PT) mode, the first quadrupole was scanned in the selected mass range and the second quadruple was used as a collision cell while the third quadrupole was fixed to monitor the ion of interest (i.e., the fatty acyl carboxylate fragmented from PE molecular species). PE molecular species including isobaric species and regiospecificity were identified by analyses of the crossed peaks with individual building blocks of PE molecular species. All mass spectral traces were displayed after normalization to the most intense peak in each individual trace. “I.S.” denotes our internal standard.

### BRIEF DESCRIPTION OF THE INVENTION

In one embodiment, a method is provided of treating a living mammal having diabetic cardiomyopathy. The method comprises administering an effective amount of an inhibitor to the mammal, performing a shotgun lipidomics analysis on the mammal and determining that the treatment was successful when and if serum or tissue biopsy cardiolipin levels are increased and/or lysocardiolipin levels are decreased.

In another embodiment, a method is provided of treating a living mammal afflicted with cardiomyopathy. The method comprises administering to the mammal an effective amount of a gene whose expression increases the synthesis of cardiolipin.

In another embodiment, a method is provided to detect diabetic cardiomyopathy in a living mammal. The method comprises analyzing a representative portion of the mammal by using shotgun lipodomics thereon to obtain and determine the cardiolipin content, comparing the obtained cardiolipin content with a cardiolipin content range previously obtained and associated for a corresponding non-diabetic mammal and determining that the analyzed mammal has diabetic cardiomyopathy when the analyzed cardiolipin content is about 5% to about 95% less than the cardiolipin content usually expected in the cells of a non-afflicted mammal.

### DETAILED DESCRIPTION OF THE INVENTION

Unless otherwise defined, technical and scientific terms used herein have the same meaning as commonly understood by one of ordinary skill in the art. Methods and materials similar or equivalent to those described herein can be used in the practice or testing of the present discovery.

All publications, patent applications, patents and other references mentioned herein are incorporated by reference in their entirety. In addition, examples are illustrative only and not intended to be limiting in any way.

As used herein, the term “gene product” includes at least to SPRR3 proteins, protein fragments, peptides, translated nucleic acid, epitopes and polypeptides thereof.

As used herein, the term “peptide” includes any of a group of compounds comprising two or more amino acids linked by chemical bonding between their respective carboxyl and amino groups. The term “peptide” includes peptides and proteins that are of sufficient length and composition to effect a biological response, e.g. antibody production or cytokine activity whether or not the peptide is a hapten. The term “peptide” includes modified amino acids, such modifications including, but not limited to, phosphorylation, glycosylation, prenylation, lipidization and methylation.

As used herein, the term “polypeptide” includes any of a group of natural or synthetic polymers made up of amino acids chemically linked together such as peptides linked together. The term “polypeptide” includes peptide, translated nucleic acid and fragments thereof.

As used herein, the term gene includes “polynucleotide” which includes nucleotide sequences and partial sequences, DNA, cDNA, RNA variant isoforms, splice variants, allelic variants and fragments thereof.

As used herein, the terms “protein”, “polypeptide” and “peptide” are used interchangeably herein when referring to a translated nucleic acid (e.g. a gene product). The term “polypeptide” includes proteins. Specifically, the term “protein” includes any large molecule composed of one or more chains of amino acids in a specific order; the order is determined by the base sequence of nucleotides in the gene that codes for the protein. The term “protein” includes a fragment and functional fragments of proteins.

As used herein, the term “nucleic acid” refers to oligonucleotides or polynucleotides such as deoxyribonucleic acid (DNA) and ribonucleic acid (RNA) as well as analogs of either RNA or DNA, for example made from nucleotide analogs any of which are in single or double stranded form.

As used herein, the term “therapeutic agent” is any molecule or atom which is conjugated, fused or otherwise affixed to an antibody moiety to produce a conjugate which is useful for therapy.

As used herein, the term “biological sample” includes vascular tissue or blood, urine or other body fluids.

As used herein a “therapeutic amount” is an amount of antibody which produces a desired or detectable therapeutic effect on or in a mammal administered the antibody.

As used herein, the term “high risk” means that there is a substantial chance and increased likelihood that the individual (living human) is afflicted with atherosclerosis. “High risk” means that there is the substantial likelihood that one or more forms of atherosclerosis is present in the human's intimae.

As used herein, the term “range of values characteristic of an individual having an increased risk of diabetes” includes those medical measurements and analytical data of a patient which are shown to be associated with the presence of atherosclerosis in patients. This range is more particularly identified by a region bordered by a low finite value and a high finite value, both values being population variable but determinable.

As used herein, the term “sample” means a viable (analyzable) sample of biological tissue or fluid. A biological sample includes an effective amount of a representative section of tissues or fluids of living animals, viable cells or cell culture.

In an aspect, the DNA or genetic construct further comprises an expression control sequence operably linked to a sequence encoding (and expressing) the expression product.

As used herein, the terms “DNA construct” or “genetic gene construct”, “gene” or “cDNA” are used interchangeably herein to, refer to a nucleic acid molecule which may be one or more of the following: regulatory regions, e.g. promoter and enhancer sequences (that are competent to initiate and otherwise regulate the expression of a gene product(s)); any other mutually desired compatible DNA elements for controlling the expression and/or stability of the associated gene product(s) such as polyadenylation sequences; other DNA sequences which function to promote integration of operably linked DNA sequences into the genome of the host cell and any associated DNA elements contained in any nucleic acid system (e.g. plasmid expression vectors) used for the propagation, selection, manipulation and/or transfer of recombinant nucleic acid sequences, sequences encoding proteins that are part of the biosensor or proteins that are functional G protein coupled receptors.

As used herein, the terms “regulatory DNA sequences” or “regulatory regions” or “DNA sequences which regulate the expression of” are used interchangeably herein to refer to nucleic acid molecules which function as promoters, enhancers, insulators, silencers and/or other similarly defined sequences which control the spatial and temporal expression of operably linked and/or associated gene products.

As used herein, the term “transgenic” refers to an organism, or progeny derived from such organism(s) by germ cell transmission or cloning, that contains exogenous genetic constructs that have been purposefully introduced into the organism. Moreover, this refers to organisms which may or may not have the introduced genetic construct stably integrated into their genome, that is, constructs which are maintained stably and can be propagated through germ cell transmission (i.e. sexual reproduction) or constructs which are expressed transiently by the organism. Furthermore, a zebrafish derived from a transgenic fish egg, sperm cell, embryo or other cell is deemed transgenic if the transgenic fish egg, sperm cell, embryo or other cell contributes DNA to the genomic DNA of the zebrafish.

As used herein the term “expression library” includes a library of chemical moieties generally whose functions are unknown. “Expression library” also includes a database, collection or assemblage of moieties or a system of containing capably identified moieties, cataloged or uncataloged, present or not present in the collection or assemblage and illustratively includes expression products of cDNA such as proteins, and enzymes including those wherein one or more of identity and function are known or unknown.

As used herein, the term “expression” includes the biosynthesis of a product as an expression product from a gene such as the transcription of a structural gene into mRNA and the translation of mRNA into at least one peptide or at least one polypeptide. The term “expression” includes gene products such as proteins and functional fragments thereof.

As used herein, the term “mammal” includes living animals including humans and non human animals such as murine, porcine, canine and feline.

As used herein, the term “isolated polypeptide” includes a polypeptide essentially and substantially free from contaminating cellular components.

As used herein, the term “isolated protein” includes a protein that is essentially free from contamination cellular components normally associated with the protein in nature.

As used herein, the term “nucleic acid” refers to oligonucleotides or polynucleotides such as deoxyribonucleic acid (DNA) and ribonucleic acid (RNA) as well as analogs of either RNA or DNA, for example made from nucleotide analogs any of which are in single or double stranded form.

As used herein, the term “patient” and

“subject” are synonymous and are used interchangeably herein.

In an aspect, pharmaceutical compositions and preparations are made in a manner well known in the pharmaceutical art. In an aspect, one preparation utilizes a vehicle of physiological saline solution comprising at least one of a chemical agent, siRNA, and penetrant combined with a pharmaceutically acceptable carrier. A suitable buffer may be present in the composition including sterile water.

In an aspect, the carrier can also contain other pharmaceutically-acceptable excipients and additives for modifying or maintaining pH, osmolarity, viscosity, clarity, color, sterility, stability, rate of dissolution, or odor of the formulation. Similarly, the carrier may contain still other pharmaceutically acceptable excipients for modifying or maintaining release or absorption or penetration.

It is also contemplated that some formulations are more conveniently administered orally in an effective amount and dosage. Such formulations are preferably encapsulated and formulated with suitable carriers in solid dosage forms.

The construction of a suitable vector can be achieved by any of the methods well-known in the art for the insertion of exogenous DNA into a vector. see Sambrook et al., 1989, Molecular Cloning, A Laboratory Manual, Cold Spring Harbor Press, N.Y.; Rosenberg et al., Science 242:1575-1578 (1988); Wolff et al., PNAS 86:9011-9014 (1989). For Systemic administration with cationic liposomes, and administration in situ with viral vectors, see Caplen et al., Nature Med., 1:39-46 (1995); Zhu et al., Science, 261:209-211 (1993); Berkner et al., Biotechniques, 6:616-629 (1988); Trapnell et al., Advanced Drug Delivery Rev., 12:185-199 (1993); Hodgson et al., BioTechnology 13:222 (1995).

This discovery is important to use an effective research tool because specific interactions are involved in almost any physiological process. If mankind is ever to cure diabetes and other debilitating diseases killing humans, thus mankind must have and use bedrock effective diagnostic and treatment tools.

Shotgun lipidomics identification of cardiolipin decrease in diabetic hearts. Calcium-independent phospholipase A₂β and iPLA₂γ were demonstrated to catalyze hydrolysis of cardiolipin as guest in phosphatidylcholine vesicles.

Since Barth's syndrome is known to result in mitochondrial dysfunction and cardiomyopathy resulting from altered cardiolipin metabolism, we hypothesized that alterations in cardiolipin were present in diabetic myocardium leading to mitochondrial dysfunction and inefficient fatty acid utilization. We exploited the recent development of shotgun lipidomics with intrasource separation and multidimensional mass spectrometry (10-12, 29) to gain insight into the types of lipid alterations present in diabetic myocardium. Through the sensitivity and specificity intrinsically inherent in these techniques, we identified the chemical entities present in lipotoxic diabetic myocardium which likely underlie lipid-mediated dysfunction in the diabetic state. Specifically, the results were remarkable for a dramatic loss of the essential mitochondrial phospholipid, cardiolipin and its direct metabolic precursor, phosphatidylglycerol.

We discovered that the depletion of phosphatidylglycerol and cardiolipin occurred in the presence of a marked decrease in glycerol-3-phosphate content likely resulting from the combined effects of the decreased glucose transport and the increased shunting of diunsaturated fatty acids (i.e., linoleic acid, the major aliphatic constituent present in cardiolipin) and other metabolic intermediates into triacylglycerols in diabetic myocardium.

Since cardiolipin is known to be essential for physiological electron transport, efficient ATP synthesis, and the function of numerous mitochondrial inner membrane enzymes and since Barth's syndrome results in mitochondrial dysfunction and cardiomyopathy, these results identify a unifying hypothesis linking altered substrate utilization and lipid metabolism with mitochondrial dysfunction and cardiomyopathy in the diabetic state.

In an aspect this discovery is used in the development of and identifidation of pharmaceuticals which would target the activation of iPLA₂β and iPLA₂γ during ischemia, diabetes, heart disease, atherosclerosis, and obesity. Biomarkers for diabetes include cardiolipin. From our discovery, inhibition of iPLA₂β and iPLA₂γ would be predicted to decrease cardiolipin degradation and improve mitochondrial function.

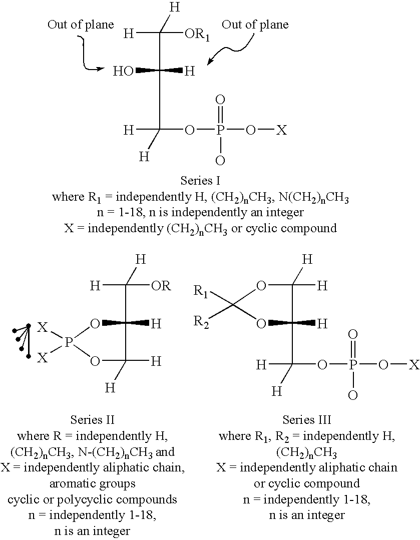

SEQUENCES:
HUMAN CARDIOLIPIN SYNTHASE
NM_019095 coding sequence

SEQUENCE NO. 1

atgctagccttgcgcgtggcgcgcggctcgtggggggccctgcgcggcgc
cgcttgggctccgggaacgcggccgagtaagcgacgcgcctgctgggccc
tgctgccgcccgtgccctgctgcttgggctgcctggccgaacgctggagg
ctgcgtccggccgctcttggcttgcggctgcccgggatcggccagcggaa
ccactgttcgggcgcggggaaggcggctcccaggccagcggccggagcgg
gcgccgctgccgaagccccgggcggccagtggggcccggcgagcaccccc
agcctgtatgaaaacccatggacaatcccgaatatgttgtcaatgacgag
aattggcttggccccagttctgggctatttgattattgaagaagatttta
atattgcactaggagtttttgctttagctggactaacagatttgttggat
ggatttattgctcgaaactgggccaatcaaagatcagctttgggaagtgc
tcttgatccacttgctgataaaatacttatcagtatcttatatgttagct
tgacctatgcagatcttattccagttccacttacttacatgatcatttcg
agagatgtaatgttgattgctgctgttttttatgtcagataccgaactct
tccaacaccacgaacacttgccaagtatttcaatccttgctatgccactg
ctaggttaaaaccaacattcatcagcaaggtgaatacagcagtccagtta
atcttggtggcagcttctttggcagctccagttttcaactatgctgacag
catttatcttcagatactatggtgttttacagctttcaccacagctgcat
cagcttatagttactatcattatggccggaagactgttcaggtgataaaa
gactga
BC069010 coding sequence

SEQUENCE NO. 3

atgctagccttgcgcgtggcgcgcggctcgtggggggccctgcgcggcgc
cgcttgggctccgggaacgcggccgagtaagcgacgcgcctgctgggccc
tgctgccgcccgtgccctgctgcttgggctgcctggccgaacgctggagg
ctgcgtccggccgctcttggcttgcggctgcccgggatcggccagcggaa
ccactgttcgggcgcggggaaggcggctcccaggccagcggccggagcgg
gcgccgctgccgaagccccgggcggccagtggggcccggcgagcaccccc
agcctgtatgaaaacccatggacaatcccgaatatgttgtcaatgacgag
aattggcttggccccagttctgggctatttgattattgaagaagatttta
atattgcactaggagtttttgctttagctggactaacagatttgttggat
ggatttattgctcgaaactgggccaatcaaagatcagctttgggaagtgc
tcttgatccacttgctgataaaatacttatcagtatcttatatgttagct
tgacctatgcagatcttattccagcgaacacttgccaagtatttcaatcc
ttgctatgccactgctag
a.a sequence for NM_019095

SEQUENCE NO. 2

mlalrvargswgalrgaawapgtrpskrracwallppvpcclgclaerwr
lrpaalglrlpgigqrnhcsgagkaaprpaagagaaaeapggqwgpastp
slyenpwtipnmlsmtriglapvlgyliieedfnialgvfalagltdlld
gfiarnwanqrsalgsaldpladkilisilyvsltyadlipvpltymiis
rdvmliaavfyvryrtlptprtlakyfnpcyatarlkptfiskvntavql
ilvaaslaapvfnyadsiylqilwcftafttaasaysyyhygrktvqvik
d
a.a sequence ofBC069010

SEQUENCE NO. 4

mlalrvargswgalrgaawapgtrpskrracwallppvpcclgclaerwr
lrpaalglrlpgigqnhcsgagkaaprpaagagaaaeapggqwgpastps
lyenpwtipnmlsmtriglapvlgyliieedfnialgvfalagltdlldg
fiarnwanqrsalgsaldpladkilisilyvsltyadlipantcqvfqsl
lchc*
Human Oleoyl-iPLA₂β

SEQUENCE NO. 5

[MQFFGRLVNTFSGVTNLFSNPFRVKEVAVADYTSSDRVREEGQLILFQN
TPNRTWDCVLVNPRDSQSGFRLFQLELEADALVNFHQYSSQLLPFYESSP
QVLHTEVLQHLTDLIRNHPSWSVAHLAVELGIRECFHHSRIISCANCAEN
EEGCTPLHLACRKGDGEILVELVQYCHTQMDVTDYKGETVFHYAVQGDNS
QVLQLLGRNAVAGLNQVNNQGLTPLHLACQLGKQEMVRVLLLCNARCNIM
GPNGYPIHSAMKFSQKGCAEMIISMDSSQIHSKDPRYGASPLHWAKNAEM
ARMLLKRGCNVNSTSSAGNTALHVAVMRNRFDCAIVLLTHGANADARGEH
GNTPLHLAMSKDNVEMIKALIVFGAEVDTPNDFGETPTFLASKIGRQLQD
LMHISRARKPAFILGSMRDEKRTHDHLLCLDGGGVKGLIIIQLLIAIEKA
SGVATKDLFDWVAGTSTGGILALAILHSKSMAYMRGMYFRMKDEVFRGSR
PYESGPLEEFLKREFGEHTKMTDVRKPKVMLTGTLSDRQPAELHLFRNYD
APETVREPRFNQNVNLRPPAQPSDQLVWRAARSSGAAPTYFRPNGRFLDG
GLLANNPTLDAMTEIHEYNQDLIRKGQANKVKKLSIVVSLGTGRSPQVPV
TCVDVFRPSNPWELAKTVFGAKELGKMVVDCCTDPDGRAVDRARAWCEMV
GIQYFRLNPQLGTDIMLDEVSDTVLVNALWETEVYIYEHREEFQKLIQLL
LSP]-CO(CH₂)₇CHCH(CH₂)₇CH₃*
*Oleoylation at any iPLA₂β residue

### EXAMPLE

The following example illustrates the best currently-known method of practicing this invention and is described in detail in order to facilitate a clear understanding of the discovery. It should be understood, however, that the detailed expositions of the application of the discovery, while indicating preferred embodiments, are given by way of illustration only and are not to be construed as limiting the discovery since various changes and modifications within the spirit of the discovery will become apparent to those skilled in the art from this description. In the following examples, which illustrate the discovery, and throughout the specification, parts and percent are by weight unless otherwise indicated.

### EXAMPLE

Materials. Synthetic phospholipids including 1,1′,2,2′-tetramyristoyl cardiolipin (T14:0 CL¹), 1,2-dimyristoleoyl-sn-glycero-3-phosphocholine (14:1-14:1 PtdCho), 1,2-dipentadecanoyl-sn-glycero-3-phosphoethanolamine (15:0-15:0 PtdEtn), 1,2-dipentadecanoyl-sn-glycero-3-phosphoglycerol (15:0-15:0 PtdGro), 1,2-dimyristoyl-sn-glycero-3-phosphoserine (14:0-14:0 PtdSer), and 1-heptadecanoyl-2-hydroxyl-sn-glycero-3-phosphocholine (17:0 lysoPtdCho) were purchased from Avanti Polar Lipids, Inc. (Alabaster, Ala.). Deuterated palmitic acid (d₃-16:0 FA) and triheptadecenoin (T17:1 TAG) were purchased from Cambridge Isotope Laboratories (Andover, Mass.) and Nu-Chek Prep, Inc. (Elysian, Minn.), respectively. Antibodies against Cytochrome c and ATP synthase β were purchased from BD Biosciences Pharmingen (San Diego, Calif.). Horseradish peroxidase linked secondary antibody and ECL™ western blotting detection reagent were from Amersham Bioscience (Piscataway, N.J.). Glycerol-3-phosphate oxidase, glycerol-3-phosphate dehydrogenase, catalase, and NADH were obtained from Roche Applied Sciences (Indianapolis, Ind.). All solvents used for sample preparation and for mass spectrometric analysis were obtained from Burdick and Jackson (Honeywell International Inc., Burdick and Jackson, Muskegon, Mich.). Glycerol-3-phosphate and other chemicals were purchased from Sigma-Aldrich (St. Louis, Mo.).
¹Abbreviation: CL, cardiolipin; ESI, electrospray ionization; m:n, acyl chain containing m carbons and n double bonds; MS, mass spectrometry; NL, neutral loss; PC, choline glycerophospholipids; PE, ethanolamine glycerophospholipids; PI, precursor ion; PlsEtn, plasmenylethanolamine(s); PtdCho, phosphatidylcholine(s); PtdEtn, phosphatidylethanolamine(s); PtdGro, phosphatidylglycerol(s); PtdInc, phosphatidylinositol(s); PtdSer, phosphatidylserine(s); SM, sphingomyelin(s); TAG, triacylglycerol; Tm:n TAG, tri m:n glycerol.

Induction of Diabetes and Sample Preparation.

Male mice (C57BL/6, 4 months of age) deliberately were purchased from The Jackson Laboratory (Bar Harbor, Me.). Diabetes was induced by a single intravenous injection (in the tail vein) of streptozotocin at 4 months of age (165 mg/kg body weight in 0.1 ml of 0.1 M citrate buffer, pH 4.5) as described previously. Control mice received citrate buffer (0.1 ml) alone. Diabetes was confirmed within 48 h by blood glucose levels >3 mg/ml as measured by chemstrips (bG, Boehringer-Mannheim).

All animal procedures were performed in accordance with the Guide for the Care and Use of Laboratory Animals (National Academy of Science, 1996) and were approved by the Animals Studies Committee at Washington University in St. Louis.

Mice were killed by asphyxiation with carbon dioxide after diabetes was induced for 4 weeks. The hearts were excised quickly and immersed in ice-cold buffer (250 mM sucrose/25 mM imidazole, pH 8.0, at 4° C.). After removing extraneous tissue and epicardial fat, each heart was quickly dried and immediately freeze-clamped at the temperature of liquid nitrogen. Myocardial wafers were pulverized into a fine powder with a stainless-steel mortar and pestle. Protein assays on the wafers were performed using a bicinchoninic acid protein assay kit (Pierce, Rockford, Ill., USA) with bovine serum albumin as a standard. A mouse myocardial sample (approximately 10 mg) was weighed from each mouse heart and lipids were extracted by the modified method of Bligh and Dyer (30) as described previously.

Internal standards including 14:0-14:0 PtdSer (1.0 nmol/mg protein), T14:0 CL (5.0 nmol/mg protein), 15:0-15:0 PtdGro (4.2 nmol/mg protein), 15:0-15:0 PtdEtn (18.75 nmol/mg protein), 14:1-14:1 PtdCho (15 nmol/mg protein), 17:0 lysoPtdCho (1 nmol/mg protein), T17:1 TAG (10 nmol/mg protein), and d₃-16:0 FA (2 nmol/mg protein) were added to each myocardial sample based on protein concentration. Thus, the lipid content can be normalized to the protein content and quantified directly. These internal standards were selected because they represent <<1% of endogenous cellular lipid molecular species present as demonstrated by ESI/MS lipid analysis without addition of these internal standards.

The extraction mixture was centrifuged at 2,500 rpm for 10 min. The chloroform layer was carefully removed and saved. To the MeOH/H₂O layer of each test tube, an additional 2 ml of chloroform was added and chloroform layer was separated as above. The chloroform extracts from each identical sample were combined and dried under a nitrogen stream. Each individual residue was then resuspended in 4 ml of chloroform/methanol (1:1), re-extracted against 1.8 ml of 20 mM LiCl aqueous solution, and the extract was dried as described above. Individual residues were resuspended in ˜1 ml of chloroform and filtered with a 0.2-μm PFTE syringe filter into a 5-ml glass centrifuge tube (this step was repeated twice). The chloroform solution was subsequently dried under a nitrogen stream and each individual residue was resuspended with a volume of 500 μl/mg of protein in 1:1 chloroform/methanol. The lipid extracts were finally flushed with nitrogen, capped, and stored at −20° C. for ESI/MS (typically analyzed within one week). Each lipid solution was further diluted approximately 50 fold just prior to infusion and lipid analysis. To the diluted lipid solutions, LiOH (50 nmol/mg of protein) was added immediately prior to performing further lipid analyses in both negative- and positive-ion modes.

Instrumentation and Mass Spectrometry.

A triple-quadrupole mass spectrometer (ThermoElectron TSQ Quantum Ultra, San Jose, Calif., USA) operating under an Xcalibur software system was utilized in this study. The first and third quadrupoles serve as independent mass analyzers while the second quadrupole serves as a collision cell for tandem mass spectrometry. The spray voltage was maintained at −3 kV in the positive-ion mode and +3 kV in the negative-ion mode. An offset voltage on the ion transfer capillary was set to 17 V and −17 V in positive- and negative-ion modes, respectively. The heater temperature along the ion transfer capillary was maintained at 250° C. The sheath gas (nitrogen) pressure was 2 psi. The diluted lipid extract solution was directly infused into the ESI source at a flow rate of 4 μl/min with a syringe pump. Typically, a 1-mm period of signal averaging in the profile mode was employed for each MS spectrum. For tandem mass spectrometry, a collision gas pressure was set at 1.0 mTorr but the collision energy was varied with the classes of lipids as described previously (29). The tandem mass spectrometry in the neutral loss (NL) mode was performed through coordinately scanning both the first and third quadrupoles with a mass difference (i.e., neutral loss) while collision activation was performed in the second quadrupole. The tandem mass spectrometiy in the precursor-ion (PI) mode was performed through scanning the first quadrupole in the interested mass range and monitoring the second quadruple with an ion of interest while collision activation was performed in the second quadrupole. Typically, a 2-min period of signal averaging in the profile mode was employed for each tandem MS spectrum. All the MS spectra and tandem MS spectra were automatically acquired by a custom sequence subroutine operated under the Xcalibur software. Data processing of two-dimensional mass spectrometric analyses including ion peak selection, data transferring, peak intensity comparison, and quantitation were conducted as previously described (29) using MicroSoft Excel Macros.

Western Blotting Analysis of Mitochondrial Proteins.

The liquid-nitrogen frozen mouse hearts harvested as described above were pulverized into a wafer powder. Lysate buffer (phosphate-buffered saline, pH 7.2, 1% Nonidet P-40, 0.5% sodium deoxycholate, 0.1% SDS, 30 μg/ml aprotinin and 0.1 mg/ml PMSF) was added at the ratio of 5 ml per gram of tissue. The tissue was disrupted and homogenized by sonication at 4° C. in a water bath sonicator for 5 mm and shearing through a 22 G needle 10 times. The homogenized tissue was incubated at 4° C. for 30 mm before being centrifuged at 10,000×g for 10 mm. The supernatant was saved and used as the protein extract from the tissue.

An equal amount of proteins from mouse heart homogenates of both diabetic and control mice were analyzed by SDS-PAGE. The separated protein bands were transferred to immobilon-P membranes. Nonspecific binding sites were blocked by powdered milk (5% (w/v)) prior to incubation with primary antibodies (anti-cytochrome c and anti-ATP synthase β. Horseradish peroxidase linked secondary antibody was used in combination with an ECL detection system to visualize immunoreactive bands.

Measurement of Glycerol-3-phosphate Mass Content in Myocardium.

Glycerol-3-phosphate was extracted from frozen mouse hearts by methanol and chloroform as previously described (31). Briefly, ice-cold methanol/chloroform (2:1, v/v) was added to the pulverized tissue at a ratio of 6:1/mg tissue. The mixture was kept on ice for 10 min to allow the tissue to thaw before addition of chloroform/water (1:2, v/v) at a ratio of 3:1/mg tissue. After sonication for 5 min at 4° C. and vortexing, the mixture was centrifuged at 14,000 g for 20 min at 4° C. The upper phase was saved and dried under a stream of nitrogen. The dried residue was resuspended into 100 μl of water and used for analysis of glycerol-3-phosphate.

Glycerol-3-phosphate content was determined by an enzymatic assay system with minor modifications (32). Specifically, 10 μl of the diluted extract or glycerol-3-phosphate standard (0-25 μM) was mixed with 40 μl of assay buffer (200 mM tricine/KOH, pH 8, 10 mM MgCl₂) and incubated at 95° C. for 20 min to destroy dihydroxyacetone phosphate. The samples were cooled on ice, centrifuged and transferred to a 96-well microplate. Additional 40 μl of assay buffer (50 mM tricine/KOH, pH 8, 10 mM MgCl₂ containing 2 units of glycerol-3-phosphate oxidase, 130 units of catalase, 0.4 units of glycerol-3-phosphate dehydrogenase, and 0.06 μmol of NADH) was added prior to incubation at 30° C. for 20 min. The absorbance at 340 nm was measured and a standard curve of the absorbance versus the concentration of NADH was derived. The amount of glycerol-3-phosphate in the sample was determined by comparing the absorbance of the sample to the standard curve.

Protein concentration was determined with a bicinchroninic acid protein assay kit (Pierce, Rockford, Ill., USA) using bovine serum albumin as a standard. Data from biological samples were normalized to the protein content and all data are presented as the mean ±SEM of a minimum of four separate animals. Statistical differences between mean values were determined by nested ANOVA analysis.

Sequences

Calcium independent phospholipase iPLA₂β

Calcium independent phospholipase iPLA₂γ

Results

Shotgun Lipidomics was performed Using 2D Mass Spectrometry to Identify Alterations of Cardiolipin Molecular Species in Control and Diabetic Mouse Myocardium.

Recent studies have underscored the role of lipotoxicity and mitochondrial dysfunction in the pathophysiology of diabetic cardiomyopathy. However, the biochemical mechanisms underlying these alterations are incompletely understood.

To determine alterations in the lipidome of murine myocardium in diabetes and their potential physiological sequelae, we examined chloroform extracts of myocardium from control and diabetic mice by recently developed shotgun lipidomics and multi-dimensional mass spectrometry approaches. First, ESI/MS spectra acquired by direct infusion of diluted mouse myocardial lipid extracts in the negative-ion mode displayed specific differences in the lipid profiles of control and diabetic mice (compare panels A and B of FIG. 1). Although similar groups of peaks were present in both the control and diabetic samples, close inspection of the first dimensional spectra identified substantial differences in the ratios of the specific peak intensities of ions (e.g., m/z 723.6) normalized to internal standards. However, examination of the first dimension alone does not identify either the specific class or molecular species which gave rise to these ion peaks.

Application of 2D mass spectrometry clearly identified the peak at m/z 723.6 crossing with a peak in the precursor-ion scanning of m/z 153 (corresponding to a phosphoglycerol derivative) and a peak in the precursor-ion scanning of m/z 279.2 (corresponding to linoleate) (FIG. 2). Thus, analyses of these peaks from the molecular ion using 2D mass spectrometry unambiguously identified the peak at m/z 723.6 as doubly-charged tetra-18:2 cardiolipin. In addition, several minor cardiolipin molecular species including 18:2-18:2-18:2-18:1 (peak at m/z 724.6 as its doubly-charged ion, the left broken line in panel A of FIG. 2), 18:2-18:2-18:2-20:4 (peak at m/z 735.6 as its doubly-charged ion, the right broken line in panel A of FIG. 2), and 18:0-18:2-18:2-20:4 ((peak at m/z 737.6 as its doubly-charged ion) were also identified. Repetitive sample analyses and comparisons of peaks with internal standards demonstrated a loss of cardiolipin mass from 7.2±0.3 nmol/mg of protein in control hearts to 3.1±0.1 nmol/mg of protein in diabetic hearts (FIG. 3A).

Mass Spectrometric Analyses of Phosphatidylglycerol, the Direct Metabolic Precursor of Cardiolipin.

Furthermore, first dimensional spectra also demonstrate substantial decrease in peak intensity of ion at m/z 747.6 in analysis of lipid extracts of diabetic mouse myocardium in comparison to that from controls (FIG. 1). Again, application of 2D mass spectrometry demonstrated that the peak at m/z 747.6 gave rise to glycerol phosphate (precursor-ion scanning of m/z 153), palmitate (precursor-ion scanning of m/z 255.2), and oleate (precursor-ion scanning of m/z 281.2) (FIG. 2). Therefore, this molecular ion was identified as deprotonated 16:0-18:1 PtdGro.

Similarly a loss of PtdGro mass from 2.5±0.2 nmol/mg of protein in control hearts to 1.3±0.1 nmol/mg of protein in diabetic heats was present (FIG. 3B). However, only modest losses of phosphatidylinositol (from 2.4±0.3 to 1.8±0.5 nmol/mg of protein) and phosphatidylserine (from 5.8±0.3 to 4.7±0.4 nmol/mg of protein) were present without any differences in molecular species distribution.

Among the PtdIns species present, the 18:0-20:4 molecular species accounts for >75% of the mass content in this class with minor amounts of 16:0-20:4, 18:1-18:1, 18:1-20:4, and 18:0-22:6 molecular species. For PtdSer molecular species, the 18:0-22:6 PtdSer accounts for ˜65% of the mass content in the class with ˜20% present as 18:0-20:4 PtdSer, and ˜15% present as 18:0-18:1 PtdSer (FIG. 2).

Assessment of Mitochondrial Inner Membrane Content in Diabetic Myocardium by Marker Enzyme Analysis.

Cardiolipin is well known to be localized to the mitochondrial inner membrane. To assess if the marked decrease in cardiolipin content in diabetic mouse myocardium are due to alterations of inner membrane mitochondrial protein mass in diabetic hearts in comparison to controls, mitochondrial marker proteins were analyzed by Western blot analysis. FIG. 4A shows representative examples of Western blot analyses of cytochrome c and ATP synthase β from myocardial homogenates of diabetic and control mice. These Western blot analyses demonstrate that both mitochondrial inner membrane marker proteins from these preparations were present in nearly equal amounts, indicating that the alterations in cardiolipin that were observed were not due to a decreased mitochondrial inner membrane content in diabetic myocardium.

Quantification of Glycerol-3-phosphate Present in Control and Diabetic Mouse Myocardium

In mammals, cardiolipin is synthesized by a series of sequential reactions that ultimately employ CDP-diacylglycerol as the electrophile with glycerol-3-phosphate acting as the nucleophile in a reaction catalyzed by phosphatidylglycerol phosphate synthase to lead to the penultimate metabolic precursor of cardiolipin. The mass spectrometric analyses unambiguously demonstrated the depletion not only of cardiolipin, but also depletion of its direct metabolic precursor phosphatidylglycerol (FIG. 3).

Since phosphatidylglycerol synthesis requires utilization of glycerol-3-phosphate and CDP-diacylglycerol for synthesis of its precursor phosphatidylglycerol phosphate, and since glucose transport and glycolytic flux are attenuated in diabetic myocardium, we hypothesized that diminished levels of glycerol-3-phosphate could contribute to the conjoint depletion of phosphatidylglycerol and cardiolipin.

Accordingly, we measured glycerol-3-phosphate levels in control and diabetic myocardium employing a spectrophotometric assay based on the conversion of NADH to NAD using an enzymatic assay system as previously described (32).

The results demonstrated that glycerol-3-phosphate decreased from 4.9±0.9 nmol/mg of protein in control myocardium to 2.2±0.3 nmol/mg of protein in diabetic myocardium (p<0.01, n=4, FIG. 4B). The data are consistent with the notion that limiting amounts of glycerol-3-phosphate contribute to the depletion of metabolites along the sequential anabolic pathway leading to cardiolipin synthesis.

Collectively, these results suggest that decreased glucose transport, metabolism or excessive removal of glycerol-3-phosphate from increased acyl CoA levels in diabetic myocardium result in a decreased availability of glycerol-3-phosphate for use in phosphatidyglycerol and cardiolipin biosynthesis in diabetic myocardium. Thus, altered substrate supply (glucose transport), decreased metabolism (glycolytic flux) and increased metabolic shunting (removal of the residual glycerol-3-phosphate to the TAG pool) each likely play roles in the critical decrease in cardiolipin mass present in diabetic myocardium. Since cardiolipin has previously been shown to be essential for efficient mitochondrial function, these results provide an integrating hypothesis linking altered substrate utilization, lipotoxicity and mitochondrial dysfunction in diabetic myocardium. We specifically point out that other mechanisms contributing to mitochondrial dysfunction are not excluded by identification of depletion of cardiolipin mass and its precursor substrates, but rather that these additional mechanisms (e.g., uncoupling by fatty acids, increased free radical oxidation, etc) could further compromise mitochondrial function in the diabetic state by additional cardiolipin depletion or through other mechanisms independent of cardiolipin depletion.

Alterations in Triacylglycerol Content and Molecular Species in Diabetic Myocardium

Traditionally, triacylglycerol accumulation in cardiac muscle has been considered a hallmark of lipotoxicity in both the obese and the diabetic states (2, 6). The direct metabolic precursors of triglycerides are diglycerides which are generated from the hydrolysis of phosphatidic acid by the action of phosphatidate phosphohydrolase. It should be noted that the conversion of phosphatidic acid to diacylglycerol serves as a key branch point in lipid metabolism facilitating entry into specific types of lipid classes and molecular species. Thus, the relative kinetics of the acylation rate of diacylglycerol (resulting from phosphatidic acid) by acyl-CoA vs. the condensation of phosphatidic acid with CTP to synthesize CDP-diacylglycerol leading to phosphatidylglycerol and cardiolipin contributes to the net amount of cardiolipin synthesis in myocardium. Accordingly, alterations in the amount and the molecular species composition of TAG can provide key information on the roles of acyl-CoA and diacylglycerol acyltransferase-mediated shunting of diacylglycerol away from cardiolipin synthesis in diabetic (diseased) myocardium.

To determine the types and amounts of TAG molecular species in “lipotoxic” diabetic myocardium, we exploited the 2D mass spectrometric analyses of acyl chain building blocks in TAG molecular species (FIG. 5). Molecular species profiles of TAG in control and diabetic myocardial lipid extracts were substantially different (FIG. 5). For example, the TAG molecular species containing arachidonate (comparison of the profiles from NL 304.3 between 2D mass spectra in FIG. 5) are dramatically different. The arachidonate-containing TAG molecular species in control mice are dominated by the ion peak at m/z 885.7 while arachidonate-containing TAG molecular species in diabetic mice shifted their maximal ion peak to m/z 909.7 with other major species were present at m/z 911.7 and 931.7.

The total mass content of arachidonate-containing TAG molecular species increased over four-fold from 0.24±0.13 nmol/mg of protein in control mice to 1.07±0.17 nmol/mg of protein. Moreover, the TAG molecular species at m/z 837.7 and 863.7 contain almost equal amounts of 16:0 acyl moieties in control myocardium whereas 16:0-containing species were predominant at m/z 863.7 and 865.7 in diabetic mice (see in the NL traces of 256.2 u in FIG. 5).

Our results demonstrated an increase in TAG mass from 5.4±0.7 nmol/mg of protein in the controls to 21.1±3.5 nmol/mg of protein in diabetic mice (Table 1). This increase of 16 nmol/mg of protein of TAG was largely accounted for by ten molecular species (16:0-18:2-18:2, 16:0-18:1-18:2, 16:11-18:1-18:1, T18:1, T18:2, 18:0-18:2-18:2, 18:1-18:2-18:2, 18:1-18:1-18:2, 16:0-18:2-22:6, and 16:0-18:1-22:6). These 10 molecular species accounted for 72% of the total increase, while the 42 other measurable TAG molecular species only accounted for 28 mol % of the increased TAG mass in diabetic myocardium (Table 1).

Identification of Increased Plasmenylethanolamines in Diabetic Myocardium

Comparison of the pseudomolecular ions present in the negative-ion EST mass spectra acquired directly from infusion of the diluted chloroform extracts of myocardium from control and diabetic mice (in the presence of LiOH) demonstrated increases in the ion peaks at m/z 746.6, 772.6, and 774.6 in diabetic myocardium which were accompanied by a concomittant decrease in the intensity of the peak at m/z 790.6 (FIG. 6). Two-dimensional mass spectrometric analyses (FIG. 7) identified the major peak at, m/z 790.6 as 18:0-22:6 PtdEtn. In diabetic myocardium, the mass of 18:0-22:6 PtdEtn decreased from 31.1±0.7 to 23.8±0.3 nmol/mg of protein (p<0.0001). Two-dimensional mass spectrometric analyses (FIG. 7) also demonstrated that ion peaks at m/z 746.6, 772.6, and 774.6 contained peaks in the precursor-ion scanning mode of m/z 327.3 (corresponding to acyl chains of 22:6). Furthermore, treatment of the lipid film with acidic vapor resulted in the disappearance of these peaks thereby demonstrating the presence of plasmalogens and identifying these peaks as 16:0-22:6, 18:1-22:6, and 18:0-22:6 PlsEtn. Comparisons with the internal standard demonstrated that these peaks correspond to 6.9±0.1, 3.5±0.1, and 6.8±0.1 nmol PlsEtn/mg of protein, respectively, in control samples. Analysis of replicate samples demonstrated that this increase in plasmenylethanolamine mass was reproducible in multiple independent preparations (20.5±1.4 mol % increase in diabetic myocardium, p<0.01, n=7).

Discussion

Recent studies have underscored the importance of alterations in lipid metabolism and resultant lipotoxicity as an underlying mechanism precipitating diastolic filling abnormalities and hemodynamic dysfunction in diabetic myocardium (2-9). In particular, the almost complete dependence on fatty acids as an energy source in diabetic myocardium is believed to contribute to mitochondrial dysfunction, although the biochemical mechanisms underlying inefficient energy production by mitochondria are not chemically defined. The 2D ESI/MS approach utilized herein led to the direct identification of a dramatic decrease in the essential mitochondrial lipid, cardiolipin and its direct biochemical precursor, PtdGro in diabetic myocardium.

In addition, shotgun lipidomics identified four-fold increases in the total mass of triglycerides, specific changes in triglyceride molecular species composition and discrete alterations in the subclass and molecular species composition of ethanolamine glycerophospholipids in diabetic myocardium. Collectively, these results identify the precise chemical entities present in lipotoxic diabetic myocardium and provide insight into the biochemical mechanisms that likely contribute to mitochondrial dysfunction and cardiomyopathy in the diabetic state.

Cardiolipin is an unusual phospholipid comprised of a dimer of phosphatidate molecules linked through a glycerol backbone. Cardiolipin is localized exclusively in the mitochondrial inner membrane where it facilitates mitochondrial function through a variety of mechanisms largely related to its highly anionic character, large aliphatic chain to polar head group volume and specific binding to proteins in the electron transport chain including cytochrome c oxidase (33-36). Depletion of cardiolipin results in release of cytochrome c from the mitochondrial inner membrane leading to the activation of cellular apoptosis through activation of caspase 8 (37, 38).

Prior work has demonstrated that palmitate overloading of cardiac myocytes induces alterations in cardiolipin content and cell death (39). Importantly, apoptosis has been implicated as an important mechanism of myocytic cell death in diabetic myopathy. Cardiolipin also promotes fusion of biological membranes due to its strong propensity to adapt an H_{II} hexagonal phase (40). In addition, the high negative charge density of cardiolipin head group may interact with different cations and influence ion channel function. The importance of mitochondrial fusion and fission in mitochondrial function are becoming increasingly appreciated (41). The essential role of cardiolipin in mitochondrial function and cardiac hemodynamics has recently been underscored through identification of a genetic disorder, Barth's syndrome, in which genetic mutations in this X-linked gene (Xq28) induce altered cardiolipin metabolism, precipitate mitochondrial dysfunction and result in a striking cardiomyopathy (24-28).

Recent studies have identified defective cardiolipin aliphatic chain remodeling in Barth's syndrome resulting in cardiolipin depletion. The causative effect of the depletion of cardiolipin content on mitochondrial and hemodynamic function are now well accepted. The pathologic decrease in cardiolipin content and altered aliphatic chain composition in Barth's syndrome has recently been shown to be initiated by the action of a mitochondrial phospholipase leading to the formation of lysocardiolipin followed by tafazzin catalyzed transacylation to yield the predominant tetralinoleoyl cardiolipin present in all known mammalian mitochondrial membranes (42). In Barth's syndrome, tafazzin enzymatic function is compromised leading to cardiolipin depletion and alterations in the molecular species content of the residual cardiolipin (43). In the diabetic state there was dramatic cardiolipin depletion but no evidence for substantial alterations in cardiolipin molecular species content (although we cannot rule out small alterations in the relative content of the low abundance (<5%) cardiolipin molecular species). From the dramatic effects of cardiolipin depletion on mitochondrial function documented in multiple laboratories, it seems reasonable to conclude that the dramatic loss of ˜60 mol % of the cardiolipin content in the inner mitochondrial membrane demonstrated by shotgun lipidomics is responsible, at least in part, for the mitochondrial dysfunction present in diabetic myocardium.

Based upon prior studies from multiple independent laboratories, the magnitude of cardiolipin depletion discovered would be predicted to result in severe defects in the kinetics and types of protein-protein interactions (e.g., complex III and complex IV supra molecular assembly) present in the mitochondrial electron transport chain. These alterations could increase uncoupling and potentially accelerate free radical generation. Moreover, the decreased efficiency of the ADP/ATP transporter (resulting in diminished chemical energy production) and decreases in the kinetics of the carnitine-acylcarnitine translocase as well as the activity of other mitochondrial inner membrane enzymes would be anticipated in the presence of this magnitude of cardiolipin depletion.

It is interesting to note that impaired function of the carnitine-acylcarnitine transporter would lead to increased levels of acylcarnitine which we and others have previously observed in diabetic myocardium (9, 44). Furthermore, the decrease in the inner membrane negative charge alters the surface properties of the mitochondrial inner membrane attenuating the efficiency of the transduction of transmembrane potential energy gradients into chemical energy (e.g., ATP). Accordingly, the 57% reduction in cardiolipin content observed likely underlies, at least in part, the mitochondrial and hemodynamic dysfunction present in diabetic myocardium.

The loss of cardiolipin mass in diabetic myocardium is likely a multifactorial process with contributions from both decreased synthesis (e.g., precursor pool depletion as demonstrated in this study) as well as increased cardiolipin degradation. Through multidimensional ESI/MS it is clearly demonstrated that PtdGro content is markedly decreased (FIG. 1) in diabetic myocardium. Since PtdGro is the direct chemical precursor of cardiolipin it seems likely that one, and perhaps a major mechanism underlying cardiolipin depletion is the markedly reduced levels of phosphatidylglycerol in diabetic myocardium. Multiple independent lines of evidence demonstrate that both glucose transport and utilization are dramatically decreased in diabetic myocardium (45-47) and our results directly identify a dramatic decrease in glycerol-3-phosphate content in diabetic myocardium. Thus, the decreased levels of glycerol-3-phosphate in diabetic myocardium likely contribute to PtdGro and cardiolipin depletion.

One potential consequence of inappropriate reliance on fatty acid substrate at the expense of glycolysis is a decreased level of glycolytic intermediates that are necessary for providing glycerol-based metabolites for multiple different lipid synthetic pathways. Finally, lipotoxicity in diabetic myocardium results in increased levels of fatty acyl-CoA which would scavenge the available glycerol-3-phosphate further amplifying this effect. One observation documenting alterations in the rate limiting steps of fatty acid utilization in mitochondria is the accumulation of acylcarnitines in diabetic myocardium we and others have previously reported (9, 48).

Prior work has demonstrated the presence of increased free radical oxidation in diabetic mitochondria which can target the bisallylic hydrogen in linoleic acid, the predominant fatty acid in mammalian cardiolipin. Furthermore, intracellular linoleic acid may be sequestered during lipotoxicity by augmentation of both its absolute and relative incorporation into triglyceride molecular species as shown by 2D ESI/MS (FIG. 5). Finally, supplementation with linoleic acid has salutary effects on restoring the physiologic molecular species of cardiolipin (49). Several types of phospholipases are located in the mitochondrial compartment and membrane-associated phospholipases are activated in the myocardium in the diabetic state (9). Intriguingly, the activation of one such mitochondrial calcium independent and bromenol sensitive phospholipase responds to alterations in the transmembrane permeability gradient which is believed to be altered in the diabetic state (50). Accordingly, it is likely that the combined contributions of decreased cardiolipin synthesis in conjunction with accelerated cardiolipin degradation are collectively responsible for the observed depletion of cardiolipin in diabetic myocardium and resultant mitochondrial dysfunction.

Cardiolipin depletion resulting in mitochondrial dysfunction could lead to the attenuated oxidation of fatty acids and resultant TAG accumulation found in diabetic myocardium. TAG molecular species increase dramatically in diabetic myocardium and have previously been implicated as the etiologic agents mediating lipotoxicity (6). TAG could serve as a source for the generation of toxic fatty acids and fatty acyl CoAs by hydrolysis by myocytic TAG lipases thereby increasing the amphiphilic burden presented to diabetic myocardium. Such alterations could affect membrane dynamics and surface charge altering electromechanical coupling and the electrophysiological properties of diabetic myocardium. In addition, TAG hydrolysis would result in the production of diacyiglycerol which could activate PKC-mediated signaling cascades and lead to dysfunctional metabolic or hemodynamic signaling cascades. Finally, it is also possible that the presence of TAG droplets could interfere with the flux of hydrophobic chemical constituents in myocytes or alter the mechanical properties of cardiac myocytes.

Cardiolipin depletion could also result in increased peroxisomal proliferation to accommodate the increased oxidative needs of myocardium. Concomitant with peroxisomal oxidation, the biosynthetic functions of peroxisomes such as ether lipid synthesis (a precursor of plasmalogen) could also increase. This study shows a remarkable increase in the mass content of plasmalogen molecular species. Plasmalogens alter the molecular dynamics of cell membranes and possess an increased dipole moment in comparison to their diacyl phospholipid counterparts (51). These alterations could also influence the electrophysiological properties of myocardium and/or calcium release during contraction or reuptake during diastolic relaxation contributing to diastolic dysfunction. It is important to note that diastolic dysfunction is an important element in diabetic cardiomyopathy and that altered calcium fluxes, in part due to changes in membrane dynamics and resultant influences on transmembrane protein activities, could play an important role.

In conclusion, cardiolipin has previously been shown to be essential for physiological electron transport, inner membrane supramolecular assembly, efficient ATP synthesis, binding of cytochrome c, and the function of multiple other mitochondrial inner membrane enzymes. The use of shotgun lipidomics has identified cardiolipin depletion and its direct metabolic precursor, phosphatidyiglycerol, thereby altering membrane charges, surface properties, and molecular dynamics. The resultant pathologic alterations in inner membrane function represent a new biochemical mechanism that integrates the altered substrate utilization present in diabetic myocardium with mitochondrial dysfunction into a unifying hypothesis. Amplification of the effects of cardiolipin depletion occurs through ineffective utilization of fatty acid substrates, resultant increases in free radical generation and accumulation of toxic amphiphilic metabolites collectively resulting in the cardiomyopathy manifest in the diabetic state.

TABLE 1
Mouse myocardial lipids were extracted using a
modified Bligh and Dyer procedure and the TAG molecular species
in the lipid extracts were identified and quantified by using the 2D ESI
mass spectrometric approach as described in the Materials and Methods.
The results are expressed in nmol/mg of protein and represent X ± SE
of seven different animals. Only the TAG molecular species that
contribute over 1% of total TAG in diabetic myocardium are listed in
the table.

| | | | |
|---|---|---|---|
| 16:0-16:0-18:2 | 837.7 | 0.07 ± 0.01 | 0.51 ± 0.12 |
| 16:0-16:0-18:1 | 839.7 | 0.06 ± 0.01 | 0.38 ± 0.02 |
| 16:0-18:2-18:2 | 861.7 | 0.45 ± 0.05 | 2.46 ± 0.41 |
| 16:0-18:1-18:2 | 863.7 | 0.27 ± 0.03 | 0.91 ± 0.16 |
| 16:1-18:1-18:1 | 863.7 | 0.37 ± 0.04 | 1.55 ± 0.31 |
| 16:0-18:1-18:1 | 865.7 | 0.15 ± 0.02 | 0.61 ± 0.11 |
| 16:1-18:0-18:1 | 865.7 | 0.04 ± 0.01 | 0.39 ± 0.09 |
| T18:2 | 885.7 | 0.48 ± 0.05 | 2.48 ± 0.40 |
| 16:0-18:0-20:4 | 885.7 | 0.11 ± 0.01 | 0.20 ± 0.02 |
| 18:1-18:2-18:2 | 887.7 | 0.63 ± 0.06 | 3.39 ± 0.63 |
| 18:1-18:1-18:2 | 889.7 | 0.25 ± 0.03 | 1.42 ± 0.31 |
| 18:0-18:2-18:2 | 889.7 | 0.15 ± 0.02 | 1.15 ± 0.25 |
| T18:1 | 891.7 | 0.13 ± 0.01 | 0.60 ± 0.06 |
| 18:0-18:1-18:2 | 891.7 | 0.03 ± 0.01 | 0.30 ± 0.03 |
| 16:0-18:2-22:6 | 909.7 | 0.24 ± 0.03 | 0.78 ± 0.13 |
| 18:2-18:2-20:4 | 909.7 | 0.03 ± 0.01 | 0.31 ± 0.09 |
| 16:0-18:1-22:6 | 911.7 | 0.25 ± 0.03 | 0.51 ± 0.06 |
| 18:1-18:2-20:4 | 911.7 | 0.07 ± 0.01 | 0.34 ± 0.04 |
| 18:0-18:2-20:4 | 913.7 | 0.10 ± 0.02 | 0.24 ± 0.06 |
| 18:2-18:2-22:6 | 933.7 | 0.21 ± 0.02 | 0.35 ± 0.06 |
| 18:1-18:2-22:6 | 935.7 | 0.31 ± 0.02 | 0.44 ± 0.04 |
| 18:1-18:1-22:6 | 937.7 | 0.19 ± 0.02 | 0.38 ± 0.04 |
| Total | | 5.38 ± 0.67 | 21.08 ± 3.27 |

Shotgun Lipidomics: Multidimensional MS Analysis of Cellular Lipidomes

Xianlin Han and Richard W. Gross

Shotgun lipidomics, comprised of intrasource separation, multidimensional mass spectrometry and computer-assisted array analysis is an emerging powerful technique in lipidomics. Through effective intrasource separation of predetermined groups of lipid classes based on their intrinsic electrical propensities, analyses of lipids from crude extracts of biologic samples can be directly and routinely performed. Appropriate multidimensional array analysis of lipid pseudomolecular ions and fragments can be performed leading to the identification and quantitation of targeted lipid molecular species. Since most biologic lipids are linear combinations of aliphatic chains, backbones and head groups, a rich repertoire of multiple lipid building blocks present in discrete combinations represent experimental observables that can be computer reconstructed in conjunction with their pseudomolecular ions to directly determine the lipid molecular structures from a lipid extract. Through this approach, dramatic increases in the accessible dynamic range for ratiometric quantitation and discrimination of isobaric molecular species can be achieved without any prior column chromatography a operator-dependent supervision. At its current gate of development, shotgun lipidomics can analyze over 20 lipid classes, hundreds of lipid molecular species and more than 95% of the mass content of a cellular lipidome. Thus, understanding the biochemical mechanisms underlying lipid-mediated disease states will be greatly facilitated by the power of shotgun lipidomics.

Expert Rev Proteomics 2(2), 253-264 (2005)

Lipidomics, the metabolomics of lipids, is a rapidly expanding field following the tremendous progress that has been made in genomics and proteomics [1, 2]. As such, lipidomics is an essential component of systems biology [3, 4]. Specifically, lipidomics is the large-scale study of organic solvent-soluble lipids by integrating many different modern techniques (e.g., mass spectrometry [MS]). The first essential step in lipidomics is to determine a total lipid profile (i.e., lipidome). The total lipid profile reflects the functional status of the cellular metabolic history and the lipid-related protein expression and functional profile of the cell resulting from metabolic, environmental or nutritional clues [5, 6]. The lipidome provides information on the biophysical state of cellular membranes [5], differences in lipid pools and turnover rates by dynamic lipidomics) [7-10], alterations in cellular energy supply [6], and lipid second messenger levels reflecting cellular metabolic responses and transcriptional programs [11]. The field of lipidomics has been greatly advanced by the development and application of MS, particularly electrospray ionization (ESI)/MS [1, 12-151]. Investigations in lipidomics are currently focused on identifying alterations in cellular and/or body fluid lipid levels indicative of pathology (e.g., the onset and progression of disease), environmental perturbations (e.g., diet, toxins or drugs), or response to treatment. Therefore, lipidomics is directly related to drug discovery and evaluation of drug efficacy in addition to its fundamental role in identifying the biochemical mechanisms of lipid metabolism and the discovery of novel biomarkers.

Cellular lipidomes are highly complex and variable, depending upon the species, cell type, internal organelles, micro-domains (e.g., rafts) and growth conditions. Furthermore, each cell type possesses different mole percentages of specific lipid classes, subclasses and molecular species (that are comprised of the differential lengths, degree of unsaturation and branching of aliphatic chains). Tens of thousands of possible lipid molecular species are predictably present in a cellular lipidome at the level of attomole to nanomole of lipids per milligram of protein. Studies in lipidomics by many investigators have focused on either one class/subclass of lipids or one of the physical/chemical properties of lipids [16-24], which is now referred to as targeted lipidomics. However, the emergence of Intrasource separation with multidimensional MS has allowed global lipid profiling and quantitation directly from crude extracts of biologic samples [1, 6, 9, 15, 25-27]. These methods have now been referred to as shotgun lipidomics, which was developed to exploit the synergy between the uses of intrasource separation and multidimensional MS.

This review provides a current summary of recent developments in shotgun lipidomics that have occurred since the authors' previous review [5]. Accordingly, this review will focus on:
Chemical principles for structure-based multidimensional mass spectrometric determination through lipid building blocks
Multiple complimentary informative dimensions for MS analyses
Conceptual and practical similarity of multidimensional MS to multidimensional nuclear magnetic resonance (NMR) in both performance and interpretation
Two-step ratiometric techniques the authors have developed for quantitation of low-abundance species by the combined use of exogenous and endogenous lipid internal standards

Collectively, these refinements in multidimensional MS for lipid analysis have advanced the technology to a new level in both its principle and application. Many recent developments in the field such as computation lipidomics [28, 29] and the new application of instrumentation [30, 31] have also been reported since the authors' previous review but are beyond the scope and focus of the present article [15].

Shotgun Lipidomics: Intrasource Separation

An essential point of ESI is the charge separation and selective ionization that separated charges undergo at a high electrical potential (typically ˜4 kV) in the ion source [32-35]. Specifically, an electrospray ion source selectively generates gas-phase cations in the positive-ion mode and results in anions in the negative-ion mode if both inherently charged moieties are present in the infused solution. If the analytes in the infused solution do not carry net inherent charge(s), these compounds can interact with small cation(s) or anion(s) available in the matrix to yield adduct ions in positive- or negative-ionmode (i.e., in conjunction with the imposed field), respectively. The ionization efficiencies of these electrically neutral compounds depend on the inherent dipoles of the compounds. The authors recognized this physical process in the electrospray ion source in their earliest study and used it to resolve lipid classes in a crude lipid extract into different categories based on the intrinsic electrical properties of each lipid class (see [1, 15] for reviews) [36]. With regard to the separation of lipid classes, this technique is analogous to using ion-exchange chromatography for separation of lipid classes (as the authors have previously employed [37]). However, this approach is rapid, direct, reproducible and avoids artifacts inherent in chromatography-based systems [38]. This new methodology has now been referred to as intrasource separation [15, 26].

Although there are tens of thousands of potential lipid molecular species present in a cellular lipidome, these species can generally be classified into three main categories based upon their electrical properties [15]. The lipid classes in the first category are those carrying at least one net negative charge under weakly acidic conditions and are therefore referred to as anionic lipids, which can be directly analyzed from diluted lipid extracts by negative-ion ESI/MS. Lipid classes in this category include cardiolipin, phosphatidylglycerol, phosphatidylinositol and its polyphosphate derivatives, phosphatidylserine, phosphatidic acid, sulfatide, acyl-CoA and anionic lysophospholipids. The lipid classes in the second category are those that are electrically neutral under weakly acidic conditions, but become negatively charged under alkaline conditions. Therefore, they are referred to as weakly anionic lipids and can be analyzed in negative-ion ESI/MS after addition of a small amount of LiOH (or other suitable bases). Ethanolamine glycerophospholipid (PE), lysoPE, nonesterified fatty acids and their derivatives, bile acids and ceramide are some examples in this category. The remaining lipid classes belong to the third category, which includes choline glycerophospholipid (PC), lysoPC, sphingomyelin, cerebroside, acylcarnitine, diacylglycerol, triacylglycerol, cholesterol and its esters. All of these lipid classes in the third category can be analyzed in positive-ion ESI/MS after addition of a small amount of LiOH to the Infused solution as lipids in the first and second categories are now anionic under these conditions. It should be pointed out that the authors generally assess the content of cholesterol and its esters in lipid extracts by employing a simple fluorometric method [39, 40]. Individual molecular species of cholesterol esters can be profiled by precursor-ion analysis as previously described [41]. Alternatively, a method to quantitate cholesterol and its derivatives by ESI tandem MS (MS/MS) after a simple one-step chemical derivatization of cholesterol to cholesterol-3-sulfate by a sulfur trioxide-pyridine complex may be employed [42].

The general strategy underlying the analyses of these categories of lipids based on this approach is illustrated in FIG. 1.1. Through these methods, a comprehensive series of mass spectra with respect to each of the aforementioned conditions can be obtained for each category of lipids (FIG. 2.1). Each ion peak in each of these mass spectra represents at least one lipid molecular species. This set of three multiplexed truss spectra effectively replace high-performance liquid chromatography (HPLC) column separation by exploiting intrasource separation. Of course, each pseudomolecular ion peak in each mass spectrum may contain nominal isobaric species resulting from either members of the same lipid class or from other class(es) in the category. Although product ion PSI/MS analyses can be performed to identify the molecular species underneath each ion peak at this stage (as the authors routinely conducted previously [15]), It is labor Intensive and the results of product ion analysis may be affected by the presence of neighboring peaks. More effective and accurate deconvolution of isobaric species can be accomplished through multidimensional MS with appropriate array analysis.

Shotgun Lipidomics: Multidimensional MS

The authors recognized that most classes of lipids in a cellular lipidome are multiple discrete covalent assemblies of a lipid backbone (typically glycerol) with linear combinations of various aliphatic chains (typically 14-22 carbons long containing variable degrees of unsaturation) with (or without) a wide variety of polar head groups (e.g., choline, ethanolamine, serine and inositol) (FIGS. 3.1 & 4.1]. Therefore, if one could effectively and unambiguously identify the presence of each building block of polar head groups and aliphatic chains (and combinations thereof) in each pseudomolecular ion, the complexities in the lipidome could be deconvoluted and readily solved. The techniques of neutral loss and precursor-ion scanning each exploits at least one of the structural characters of these building blocks to provide the tools to efficiently profile each ion peak after army construction, and deconvolution to identify the building blocks present and identify the moieties from which they were derived. Following this concept, a new technique, referred to as multidimensional MS, has recently been developed [1, 9, 15, 25-27].

A coordinated series of sequential 2D mass spectra are the basic components of multidimensional MS. In the first dimension, each 2D mass spectrum contains the primary (molecular or pseudomolecular) ions in the x axis of mass-to-charge ratio (m/z) while the second dimension, in most cases, is comprised of the individual building blocks (i.e., polar head groups and/or aliphatic chains) of lipids (which are characterized by either neutral loss scanning or precursor-ion scanning or both) in an axis of mass (in the rare of neutral loss scanning) or m/z (in the case of precursor-ion scanning) (FIG. 5.1). One feature of a 2D mass spectrum is that each imaginary mass spectrum along a vertical line through each m/z of the primary ion (see the broken lines in FIG. 5.1) represents a pseudo product ion mass spectrum of a precursor ion at the primary ion mass spectrum crossed with the broken line. This series of arrayed spectra is entirely analogous to a 2D-NMR spectroscopy where axes are comprised of distinct frequency domains.

Each 2D-ESI mass spectrum predictably varies with different:
Infused solution conditions (e.g., lipid concentration, acidic/alkaline condition and solvent polarity, which can be readily achieved by installation of a mixer in the front of a spray capillary tube and can be controlled by operational software)
Ionization conditions (e.g., source temperature and spray voltage)
Fragmentation conditions (e.g., collision gas pressure, collision energy, collision gas and MS/MS scanning modes/settings)

These points aid in the identification, quantitation and study of lipids. Each of these variables facilitates the construction of additional dimensions that can be built upon each 2D mass spectrum foundation, which collectively constitutes a new level of information directly obtainable from lipid mass spectrometric analysis (i.e., multidimensional MS). Specifically, multidimensional MS is defined as the aggregate of mass spectrometric analyses conducted under a variety of instrumental variables that collectively comprise an n-dimensional spectrum. Each of these variables forms one dimension of the multidimensional mass spectrum from which a 2D mass spectrum can be constructed for ease of use and display. For example, FIG. 6.1 shows a 2D-ESI mass spectrum of neutral loss of 50.0 u (i.e., loss of chloromethane from the chlorine adducts of phosphocholine-containing molecular species) from a diluted hepatic lipid extract under conditions with a variety of collision energies in the second dimension. This 2D mass spectrum illustrates the differential fragmentation kinetics of chlorine adducts of hepatic PC and sphingomyelin molecular species.

2D mass spectrometric analysis for the identification of lipid building blocks is different from MS/MS analysis, although a 2D mass spectrum for building block analysis includes of a collection of MS/MS spectra from neutral loss and/or precursorion scanning of numerous precursor ions in its arrayed format. The 2D mass spectrum for building block analysis exploits array analysis techniques integrating both the primary ion mass spectrum and associated neutral loss/precursorion spectra to determine molecular composition and amount of a lipid constituent from a single automated platform. As previously mentioned, one very important feature of a 2D mass spectrum is the presence of pseudo product-ion mass spectra for each pseudomolecular ion in the primary ion mass spectrum. Therefore, many of the characteristics of product-ion analysis can be extracted from the 2D mass spectrometric analysis. Regiospecific identification of each individual molecular species [43] and quantitative analysis of isobaric species are two important features of product-ion analyses (among others) that can be readily achieved in 2D-MS analysis [9, 26]. Another very important feature of a 2D mass spectrum is the increase of dynamic range relative to a selected internal standard. Therefore, quantitation and refinement of low-abundance molecular species with a selected internal standard for each lipid class can also be readily achieved by 2D [26, 27], but not by MS/MS analyses where a set of internal standards must be employed [44-47]. Most Importantly, identification and quantitation of each individual molecular species by multidimensional MS can be automated, and thus multidimensional MS analysis of lipids represents a high-throughout platform for global studies of the cellular lipidome.

Shotgun Lipidomics: Quantitation of Individual Molecular Species

Accurate quantitation of each individual molecular species can be achieved by multidimensional MS through a two-stage ratiometric process [26, 27]. First, the abundant molecular species in a class are quantitated by comparison with a preselected internal standard for the lipid class in the 1D (primary ion) mass spectrum. Next, these quantified values are used as endogenous internal standards in combination with the original exogenous internal standard for ratiometric comparisons. This is performed to quantitate or reline the mass content of low-abundance individual molecular species from at least one representative MS/MS scan for the class of interest in the 2D-MS for building block analyses.

The key advantage in this two-stage process is the increase of dynamic range. There are many different measures of dynamic range that it affords. For example, the dynamic range of concentration in which the quantitative technique is linear. This is the most commonly accepted meaning of the concept for dynamic mange in the literature. The authors have demonstrated this measure of dynamic mange in the low lipid concentration range in many of their studies [25, 36, 48, 49]. Another measure of dynamic range is the relative ratio of internal standard versus individual molecular species of interest. A 100-fold dynamic range (from 0.1 to 10 of the ratio) can generally be achieved. However, this dynamic range can suffer by the presence of background noise (i.e., chemical noise) and baseline drift (i.e., instrumental stability) in some cases. Therefore, under adverse experimental conditions, low-abundance molecular species can only be approximated (or not quantitated at all) and require 2D analyses. Through MS/MS in a 2D-MS format, a 400-fold (even up to 1000-fold as long as the concentration measures of dynamic range are linear over 1000-fold in comparison with controls) increase can be obtained. The authors find that this dynamic range can be achieved in almost all cases since background noise is dramatically reduced and different intensity peaks of the sane class can be found in the primary ion spectra to serve as ratiometric makers for the quantitation of low-abundant molecular species. However, the authors specifically point out that these conditions must be validated and that additional internal standards ray need to be employed in rare cases.

The main advantage of this approach for the quantitation of individual lipid molecular species in each lipid class is its simplicity in comparison with the quantitation of lipid species by MS/MS (in which multiple internal standards for each lipid class must be selected to eliminate the effects of acyl chain length, degrees of saturation and double bond locations on the kinetics of pseudomolecular ion fragmentation [44, 45, 50, 51]). The authors have demonstrated that the response factors of individual molecular species in most of the polar lipid classes rarely depend on the physical properties of aliphatic chains in biologic samples, but rather on the electrical properties of the polar head groups (i.e., dipole moments) under conditions that utilize low concentrations of lipids (<10 pmol/μl) so that aggregates do not form in 1:1 CHCl3/MeOH 36,48,521. In all cases, corrections for any differences in 13C isotopomer peak intensities must be made for accurate results [15, 25]. In this study, the authors further examined the response factors of 11 PC molecular species that possess different aliphatic chains in equimolar mixtures of 1 pmol/μl or less (each) and found that the response factors of these PC species were essentially identical within experimental error after correction for different ¹³C isotopomer distribution (FIG. 7). These results demonstrate that individual molecular species of a polar lipid class can be quantitated using one internal standard for the class. Somerharju and colleagues independently examined the effects of acyl chain length, unsaturation and lipid concentration on the response factors of instruments and found that the response factors were similar within experimental errors in the low lipid concentration region, supporting the authors' previous observations [53]. Furthermore, if their data were corrected to account for the different ¹³C isotopomer distributions, the response factors of molecular species containing different acyl chain lengths would then yield virtually identical results to those previously described [25, 49]. Thus, the response factors of individual molecular species in a polar lipid class rarely depend on the physical properties of acyl chains in the low concentration range as determined in independent laboratories.

A set of endogenous internal standards from a given class in addition to the original external standard are generally well distributed in biologic samples regarding different aliphatic chain lengths and degrees of unsaturation. Therefore, these endogenous standards represent superior standards to human-selected internal standards for lipid quantitation by MS/MS where the overlap of added internal standard ions with endogenous molecular ions must be considered, thereby limiting the candidates that can be selected for exogenous internal standards. One weakness present in 2D-MS analysis of lipids to quantitate and/or refine low-abundance molecular species is that the endogenous set of standards are secondary to the original internal standard and thus the experimental errors of the mass content of these low-abundance molecular species are amplified. However, the total mass content of these low-abundance molecular species typically only account for less than 5 mol % of the entire mass of the class. Therefore, the amplified experimental error for the mass content of these low-abundance species will not substantially affect the accuracy of quantitation for the entire class of lipids. The authors would also like to point out that the peaks composed of multiple isobaric molecular species should not be selected as an endogenous internal standard to minimize the effects of differential fragmentation on quantitation. as previously discussed [15].

Applications

The first application of shotgun lipidomics after intrasource separation and multidimensional MS was the quantitation and fingerprinting of triacylglycerol (TAG) molecular species directly from a crude lipid extract of a biologic sample [25]. Since there is no polar head group present in TAG molecular species, the second dimension of a 2D mass spectrum for TAG analysis represents the building blocks of TAG aliphatic chains that can readily be identified by neutral loss scanning of all naturally occurring fatty acids from lithiated or sodiated TAG molecular ions as previously described [25, 54-56]. One important feature of this methodology for TAG analysis is the ease in identifying individual isobaric TAG molecular species due to the abundance of multiple TAG molecular species present at each m/z value in lipid extracts of biologic samples. To date, this methodology represents the most sensitive, accurate and efficient technique for individual TAG molecular species analysis. This method has been extensively used in biologic, pathologic and pathophysiologic studies in the last 3 years (e.g., [9, 11, 26, 57-60). It should be emphasized that the location of double bonds in the constituent acyl chains is not identified by this method. However, if that is desirable, the regiospecificity of acyl chains in TAG species maybe identified in multidimensional MS by varying collision energy.

Recently, multidimensional ESI/MS has been used to identify the critical role of peroxisomal processing of fatty acids in adipocyte lipid storage and metabolism [9]. 2D-ESI/MS analyses demonstrated the accumulation of old chain length unbranched fatty acids in all major lipid classes in 3T3-L1 differentiating adipocytes, indicating the rapid a-oxidation of unbranched fatty acids. Further studies identifying the double bond location in odd chain length unbranched fatty acids found the exclusive presence of Δ9 olefinic species, suggesting the presence of two critical processes in fatty acid handling in adipocyte lipid storage and metabolism. First, monounsaturated fatty acids (e.g., oleic and palmitoleic acids) are not subject to α-oxidation, resulting in the absence of Δ8 unsaturated odd chain length fatty acids. Second, α-oxidation of saturated fatty acid substrate obeys the obligatory sequential ordering of α-oxidation prior to Δ9 desaturation [9].

Very recently, 2D-MS analysis has been exploited to investigate the energy mobilization in modest caloric restriction in mice and the mobilization of lipids in this process. Remarkably, only brief periods of fasting (4 and 12 h) result in multiple specific changes in the murine myocardial lipidome [6]. Specifically, substantial and specific depletion of PC and PE species containing polyunsaturated acyl chains occurred in murine myocardial, accounting for a total decrease of 39 nmol/mg protein in these pools after 12 h fasting and representing approximately 25% of total phospholipid mass and approximately 20 cal of Gibbs free energy/g wet weight of tissue. Furthermore, other myocardial phospholipid pods such as phosphatidylserine and phosphatidylinositol were not altered after fasting. No decrease in TAG mass was observed in myocardium during fasting; however, during 12 h of refeeding, myocardial TAG increased nearly threefold and returned to baseline levels after 24 h of refeeding. In contrast to the lipid alterations in myocardium, no changes in phospholipid mass were present in skeletal muscle and a dramatic decrease in skeletal muscle (or skeletal muscle associated) TAG mass was prominent after 12 h of fasting. These results identify phospholipids as a rapidly mobilizable energy source during modest caloric deprivation in murine myocardium while TAGs are a major source of energy reserves in skeletal muscle.

### SUMMARY & CONCLUSION

Shotgun lipidomics, based on intrasource separation. multidimensional MS and array analysis, has recently emerged as a powerful technique in the direct analysis of global cellular lipidomes. Intrasource separation can largely replace ion-exchange chromatography steps, allowing resolution of lipid classes based on the electrical properties of individual lipid classes. Multidimensional MS analysis facilitates an efficient identification of each subsequent individual molecular ion peak including potential nominal isobaric molecular species as well as the polar head groups, acyl moieties and the regiospecificity of each molecular species. The two-step quantitation process in 2D-MS for the analysis of building blocks provides an expanded dynamic range relative to a selected internal standard for each lipid class and represents an efficient and accurate method to quantify individual lipid molecular species. At the current stage of shotgun lipidomics, the analyses of over 20 lipid classes, hundreds of lipid molecular species and greater than 95% of the mass content of a cellular lipidome can be readily achieved. Its broad applications in biologic, pathologic and pathophysiologic studies have demonstrated the power and utility of shotgun lipidomics. It is anticipated that identification of many biochemical mechanisms underlying lipid metabolism critical to disease states will be uncovered through the use of shotgun lipidomics.

Expert Opinion

One key step to successfully perform shotgun lipidomics is the preparation of the sample. Commonly, crude lipid extracts are prepared by the Folch method [61] or the modified method of Bligh and Dyer [62]. Small residual aqueousphase contaminants in the extracts is inevitable, and thus back extraction or multiple extractions against an aqueous phase with a low salt concentration should be used to remove aqueous-soluable contaminants that adversely effect spectral quality. Correct pH and ionic strength conditions must be employed during sample preparation since acidic conditions in the aqueous phase can improve the extraction efficiency for acidic lipids (e.g., PtdH and acyl CoA) while destroying others (note that vinyl ether-containing compounds [i.e., plasmalogens] are acid labile). in addition, acidic/alkaline conditions must be strictly maintained to facilitate the selectivity of intrasource separation. The authors have found that lipid extraction against a low concentration LiCl solution (a weakly acidic condition) represents a suitable condition for extracts of most tissues, fluids and cells [15]. Since extraction recoveries of different lipid classes can vary, it is recommended to re-extract multiple times (at least twice) to afford a nearly complete extraction of all relevant lipids. The authors have found that the effects of differences in molecular species in a class on the extraction recoveries of these species are quite small. in addition, it is emphasized that the internal standard for each of the lipid classes should be added prior to lipid extractions for lipid analyses. Accordingly, the extraction recoveries of lipid classes are accounted for by comparisons with internal standards even if a complete extraction cannot be achieved.

Response factors of different molecular species in a class depend on the physical properties (i.e., length and saturation) of aliphatic chains to only a small degree after correction of isotopomer content when experiments are performed in the appropriate (low) concentration region [38, 53, 63]. The first consideration is the lipid concentration of the infused solution. Lipids, unlike other analytes, are unique in terms of their high hydrophobicity. When concentrations of lipids increase, they tend to aggregate to form micelles, even in some organic solvents [64]. It is well known that the longer the chain length and the higher the degrees of saturation of a lipid species, the lower the critical micellar concentration of the compound. Therefore, molecular species containing short acyl chains and/or polyunsaturation might show higher apparent response factors than those containing long and/or saturated acyl chains at a high lipid concentration if the lipid concentration exceeds approximately 10 pmol/μl [53, 63]. However, at low concentrations (<10 pmol/μl) in 1:1 (v/v) of chloroform/methanol, lipid-lipid interactions are rare and ionization efficiency of lipid mixtures largely depends on the electrical properties of each lipid molecular species, which is predominantly determined by the dipole in the polar head groups. Therefore, identical response factors for different molecular species in a class can be obtained and have been repeatedly and independently verified by multiple groups [36, 38, 53, 65]. However, when the concentration of lipids in the infusion solution increases to the point where lipid-lipid interactions become apparent, these response factors are no longer identical. Thus, concentration of lipids by straight or reversed phase chromatography must be performed with extreme caution since it promotes lipid-lipid interactions [38]. The maximal concentrations of lipids at which lipid-lipid interactions are small evidently depend on the solvent components used in the infusion solution. Therefore, a solvent system containing water, acetonitrile or a high percentage of methanol is not favored for global lipid analysis by shotgun lipidomics, although such a solvent system may be used for the analysis of a specific class of lipids by ESI/MS. The second crucial consideration is the different ¹³C isotopomer intensity distributions as described previously 15, 25, 49]. These effects could cause considerable differences between the apparent response factors of different molecular species and must be corrected in comparison with a selected internal standard as shown in FIG. 7.1. Alternatively these effects can be eliminated by determining the peak intensities after a deisotope calculation.

Caution should be exercised in employing ESI/MS/MS for quantitation of individual molecular species of each class of lipids, since the fragmentation patterns of each lipid molecular species depend on both the applied energy for collision-induced dissociation and on the structure of individual molecular species (FIG. 6.1 [25, 38, 43, 49, 66]. Changes in applied collision energy alter the kinetics of individual fragmentation pathways and result in changes in the distribution of the observed fragment ions. Thus, it is important to closely control fragmentation energies and to utilize both appropriate internal standards for each lipid class and molecular species as well as ratiometrically quantify each individual species so that identical physical parameters are compared.

Five-Year View

Shotgun lipidomics is a rapidly evolving technology. The authors believe the techniques described herein will be extended to identify low-abundance concentration lipid classes through the integration of enrichment techniques (e.g., nano-HPLC) and the development of new MS/MS methods for the identification of these classes. Additionally, the development of instruments with greatly improved sensitivity and resolution will extend penetration into the low-abundance region of cellular lipidomes. To this end, enrichment approaches in conjunction with ESI Fourier transform ion cyclotron resonance MS holds much promise [13]. Second, high-efficiency direct-infusion techniques such as microfluidic approaches will be integrated into shotgun lipidomics to accommodate the need for high through put. Third, bioinformatics in lipidomics through database development and automation of data processing will play an essential role in the development and utility of shotgun lipidomics. Finally, it appears likely that affordable robust platforms for shotgun lipidomics will be made available to the biomedical research community for even routine clinical applications such as diagnosis and monitoring of drug therapy. The authors speculate that the large flux of quantitative lipidomics data integrated with genomic and proteomic studies will significantly enhance our understanding of the role of lipids in biologic systems. Advances in this field may also lead to enhanced diagnosis of lipid-related disease states at earlier time points to enhance therapeutic efficacy and tailor drug therapy in the next 5 years.

In one embodiment, a method is provided of screening to identifying a pharmaceutical useful as a penetrant which comprises administering a candidate penetrant to a living mammal which has determined to be afflicted with cardiomyopathy, analyzing a representative sample of the mammal for cardiolipin content and determining that the candidate is a penetrant when the cardiolipin content increases and/or the cardiomyopathy is ameliorated. In an aspec the candidate is taken from a library of chemicals. In an aspect the library candidate increases expression of cardiolipin or an enzyme producing an intermediate in cardiolipin synthesis. In an aspect the library comprises compounds including at least one compound selected from Series I, II, and III glycerophosphate derivatives hereinafter depicted.

In one embodiment, a method is provided to capably detect the condition of diabetic cardiomyopathy present in a living mammal. The method comprising analyzing a representative portion of said subject by using shotgun lipodomics thereon to obtain and determine the cardiolipin content, comparing the obtained cardiolipin content with a cardiolipin content range previously obtained and associated for a non diabetic afflicated corresponding mammal and determining that the analyzed mammal has diabetic cardiomyopathy when the analyzed cardiolipin content is about 5% to about 95% less than the cardiolipin content that usually expected in the cells of a non afflicted mammal.

In one embodiment, a biomarker useful for determining the presence of diabetic cardiomyopathy in a living mammal is provided. The biomarker comprises a decreased cardiolipin content which is about 5% to 95% of that of a nonafflicted corresponding mammal.

In one embodiment, a method is provided of treating a living mammal afflicted with a diabetic cardiomyopathy. The method comprises administering a penetrant to the mammal in an amount effective to enable the penetrant to enter a cell membrane wherein the penetrant (a) self metabolizes to a species active to function as a glucose surrogate or (b) can be metabolized by action of a moiety in an amount sufficient to induce the metabolite product to capably function as a glucose surrogate in a living mammal cell.

In one embodiment, a method is provided of screening to identifying a pharmaceutical useful as a penetrant. The method comprises administering a candidate penetrant to a living mammal which has determined to be afflicted with cardiomyopathy, analyzing a representative sample of the mammal for cardiolipin content and determining that the candidate is a penetrant when the compound is internalized and converted to glycerophosphate and/or a moiety capable of being enzymatically metabolized.

In one embodiment, a method is provided of treating humans for diabetes. The method comprises administering an effective amount of a penetrant to a cell membrane. The penetrant having a functional characteristic of (a) self metabolizing to a moiety which capably functions as a glucose surrogate in a cell environment of a diabetic living mammal or (b) is capable of being metabolized to a moiety which capably functions as glucose surrogate in a cell environment of a diabetic living mammal.

In one embodiment, a glucose surrogate fluid composition is provided which comprises water, and a cell membrane penetrant which when present in the living environment of the mammal cell functions as a functional glucose surrogate.

In one embodiment, an isolated and characterized oleoyl-iPLA₂β has Sequence ID NO. 5. In an aspect, the oleoyl-iPLA₂β comprises that of a living human. In an aspect, the oleoyl-iPLA₂β comprises that similar to that in a living mouse.

In one embodiment, a process is provided for screening diabetic biomarkers. The process comprises analyzing a sample of biomass known to be free of diabetes by lipidomics, analyzing a sample of living biomass suspected of having diabetes for alterations in lipid context, comparing the analysis of the first sample with the analysis of the second sample and concluding that the second sample is indicative of the presence of diabetes in the living animal source when alterations in lipids, particularly found in cardiolipin anabolic or catabolic pathways are detected. In an aspect the analysis comprises an analysis for lipids.

In one embodiment, a target for pharmacological research comprises screening libraries for effects on cardiolipin synthesis characterized in that the target will show changes when a candidate drug active to either anabolic or catabolic enzymes is presented thereto.

In one embodiment, a method is provided of doing pharmacological research to identify compounds that effect the amount or activity of enzymes or intermediates. The method comprises effectively presenting a candidate drug to a living animal wherein the drug is capably and effectively presented to an assay system in vitro or in vivo as a target and detecting the same and concluding that alterations in cardiolipin mass or intermediates have occurred.

In one embodiment, a method is provided of identifying those living animals having diabetes. The method comprises analyzing a representative sample of living tissues from the animal for cardiolipin, cardiolipin metabolizing enzymes or cardiolipin intermediates and comparing the results of that analysis with normal controls, determining that the animal has diabetes when statistically significant alterations are found.

In one embodiment, a method is provided of determining an activator or inhibitor of iPLA₂β through modulation of calcium-dependent calmodulin inhibition. The method comprises developing and testing compounds which will modulate the interaction between iPLA₂β and calmodulin, resulting in an increase or decrease in iPLA₂β phospholipase A₂ activity.

In one embodiment, a method is provided of developing or modifying structural cogeners of fatty acyl-CoAs to allow cell permeability and stability within the cell (non-hydrolyzable analogs) which will mimic the action and mechanism of CIF (calcium-influx factor) through activating iPLA₂β by reversing the calmodulin inhibition of the enzyme.

In another embodiment, a method is provided of medically treating a mammal. The method comprises administering a pharmacological compound (drug or pharmaceutical which activates iPLA₂β from its inhibitory complex with calmodulin or inhibits iPLA₂β by stabilizing the inhibitory complex with calmodulin) in therapeutically effective amounts as an activator or inhibitor to the mammal.

The article Global analyses of cellular lipidomes directly from crude extracts of biological samples by ESI mass spectrometry: a bridge to lipidomes, Xianlin Han, Richard W. Gross, Washington University in St. Louis, Lipid Research, Inc. is incorporated herein in its entirety by reference. Volume 44, 2003, used with permission.

This work was supported by NIH grant PO1HL57278 and RO1HL41250 as well as the U.S. Neurosciences Education and Research Foundation.

While the invention has been described in terms of various specific embodiments, those skilled in the art will recognize that the invention can be practiced with modification within the spirit and scope of the claims.

### REFERENCES

Kudo, I., and Murakami, M. (2002) Prostaglandins Other Lipid Mediat 68-69, 3-58.
Scott, D. L., and Sigler, P. B. (1994) Adv Protein Chem 45, 53-88.
Tischfield, J. A. (1997) J Biol Chem 272, 17247-17250.
Leslie, C. C. (1997) J Biol Chem 272, 16709-16712.
Underwood, K. W., Song, C., Kriz, R. W., Chang, X. J., Knopf, J. L., and Lin, L. L. (1998) J Biol Chem 273, 21926-21932.
Pickard, R. T., Strifler, B. A., Kramer, R. M., and Sharp, J. D. (1999) J Biol Chem 274, 8823-8831.
Chiba, H., Michibata, H., Wakimoto, K., Seishima, M., Kawasaki, S., Okubo, K., Mitsui, H., Torii, H., and Imai, Y. (2004) J Biol Chem 279, 12890-12897.
Andrews, D. L., Bearnes, B., Summers, M. D., and Park, W. D. (1988) Biochem J 252, 199-206.
Wolf, M. J., and Gross, R. W. (1996) J Biol Chem 271, 30879-30885.
Tang, J., Kriz, R. W., Wolfman, N., Shaffer, M., Seehra, J., and Jones, S. S. (1997) J Biol Chem 272, 8567-8575.
Mancuso, D. J., Jenkins, C. M., and Gross, R. W. (2000) J Biol Chem 275, 9937-9945.
Schaffer, J. F., and Lodish, H. F. (1994) Cell 79, 427-436.
Hall, A. M., Smith, A. J., and Bernlohr, D. A. (2003) J Biol Chem 278, 43008-43013.
Marszalek, J. R., Kitidis, C., Dararutana, A., and Lodish, H. F. (2004) J Biol Chem 279, 23882-2389 1.
Smith, S., Witkowski, A., and Joshi, A. K. (2003) Prog Lipid Res 42, 289-3 17.
Moon, Y. A., and Horton, J. D. (2003) J Biol Chem 278, 7335-7343.
Dircks, L., and Sul, H. S. (1999) Prog Lipid Res 38, 461-479.
Coleman, R. A., and Lee, D. P. (2004) Prog Lipid Res 43, 134-176.
Hiltunen, J. K., and Qin, Y. (2000) Biochim Biophys Acta 1484, 117-128.
Eaton, S., Bartlett, K., and Pourfarzam, M. (1996) Biochem J 320 (Pt 2), 345-357.
Mannaerts, G. P., Van Veldhoven, P. P., and Casteels, M. (2000) Cell Biochem Biophys 32 Spring, 73-87.
Kakar, S. S., Huang, W. H., and Askari, A. (1987) J Biol Chem 262, 42-45.
Branstrom, R., Leibiger, I. B., Leibiger, B., Corkey, B. B., Berggren, P. O., and Larsson, O. (1998) J Biol Chem 273, 31395-31400.
Gribble, F. M., Proks, P., Corkey, B. B., and Ashcroft, F. M. (1998) J Biol Chem 273, 26383-26387.
Glick, B. S., and Rothman, J. E. (1987) Nature 326, 309-3 12.
Pfanner, N., Orci, L., Glick, B. S., Amherdt, M., Arden, S. R., Malhotra, V., and Rothman, J. E. (1989) Cell 59, 95-102.
Ostermann, J., Orci, L., Tani, K., Amherdt, M., Ravazzola, M., Elazar, Z., and Rothman, J. E. (1993) Cell 75, 1015-1025.
Fitzsimmons, T. J., McRoberts, J. A., Tachiki, K. H., and Pandol, S. J. (1997) J Biol Chem 272, 31435-31440.
Knudsen, J., Jensen, M. V., Hansen, J. K., Faergeman, N. J., Neergaard, T. B., and Gaigg, B. (1999) Mol Cell Biochem 192, 95-103.
Linder, M. E., and Deschenes, R. J. (2003) Biochemistry 42, 4311-4320.
Hertz, R., Magenheim, J., Berman, I., and Bar-Tana, J. (1998) Nature 392, 512-516.
Rajas, F., Gautier, A., Bady, I., Montano, S., and Mithieux, G. (2002) J Biol Chem 277, 15736-15744.
Elholm, M., Dam, I., Jorgensen, C., Krogsdam, A. M., Holst, D., Kratchmarova, I., Gottlicher, M., Gustafsson, J. A., Berge, R., Flatmark, T., Knudsen, J., Mandrup, S., and Kristiansen, K. (2001) J Biol Chem 276, 21410-21416.
Murakami, K., Ide, T., Nakazawa, T., Okazaki, T., Mochizuki, T., and Kadowaki, T. (2001) Biochem J 353, 231-238.
Forman, B. M., Chen, J., and Evans, R. M. (1997) Proc Natl Acad Sci USA 94, 4312-4317.
Kliewer, S. A., Sundseth, S. S., Jones, S. A., Brown, P. J., Wisely, G. B., Koble, C. S., Devchand, P., Wahli, W., Willson, T. M., Lenhard, J. M., and Lehmann, J. M. (1997) Proc Natl Acad Sci USA 94, 43 18-4323.
Xu, H. E., Lambert, M. H., Montana, V. G., Parks, D. J., Blanchard, S. G., Brown, P. J., Sternbach, D. D., Lehmann, J. M., Wisely, G. B., Willson, T. M., Kliewer, S. A., and Milburn, M. V. (1999) Mol Cell 3, 397-403.
Hunt, M. C., and Alexson, S. B. (2002) Prog Lipid Res 41, 99-130.
Gross, R. W. (1983) Biochemistry 22, 564 1-5646.
Nocito, M., Roy, G., Villar, L. M., Palacios, C., Serrano, A., Alvarez-Cermeno, J. C., and Gonzalez-Porque, P. (1996) Biochim Biophys Acta 1299, 17-22.
Camp, L. A., Verkruyse, L. A., Afendis, S. J., Slaughter, C. A., and Hofmann, S. L. (1994) J Biol Chem 269, 23212-23219.
Soyombo, A. A., and Hofmann, S. L. (1997) J Biol Chem 272, 27456-27463.
Duncan, J. A., and Gilman, A. G. (1998) J Biol Chem 273, 15830-15837.
Hertz, R., Kalderon, B., Byk, T., Berman, I., Za'tara, G., Mayer, R., and Bar-Tana, J. (2005) J Biol Chem 280, 24451-24461.
Lehman, J. J., Brown, K. A., Ramanadharn, S., Turk, J., and Gross, R. W. (1993) J Biol Chem 268, 207 13-20716.
Wolf, M. J., Wang, J., Turk, J., and Gross, R. W. (1997) J Biol Chem 272, 1522-1526.
Atsumi, G., Tajima, M., Hadano, A., Nakatani, Y., Murakami, M., and Kudo, I. (1998) J Biol Chem 273, 13870-13877.
Ramanadham, S., Hsu, F. F., Zhang, S., Jin, C., Bohrer, A., Song, H., Bao, S., Ma, Z., and Turk, J. (2004) Biochemistry 43, 918-930.
Roshak, A. K., Capper, E. A., Stevenson, C., Eichman, C., and Marshall, L. A. (2000) J Biol Chem 275, 35692-35698.
Su, X., Mancuso, D. J., Bickel, P. E., Jenkins, C. M., and Gross, R. W. (2004) J Biol Chem 279, 21740-21748.
Ramanadham, S., Song, H., Hsu, F. F., Zhang, S., Crankshaw, M., Grant, G. A., Newgard, C. B., Bao, S., Ma, Z., and Turk, J. (2003) Biochemistry 42, 13929-13940.
Smani, T., Zakharov, S. I., Leno, E., Csutora, P., Trepakova, E. S., and Bolotina, V. M. (2003) J Biol Chem 278, 11909-11915.
Smani, T., Zakharov, S. I., Csutora, P., Leno, E., Trepakova, E. S., and Bolotina, V. M. (2004) Nat Cell Biol 6, 113-120.
Mancuso, D. J., Abendschein, D. R., Jenkins, C. M., Han, X., Saffitz, J. E., Schuessler, R. B., and Gross, R. W. (2003) J Biol Chem 278, 2223 1-22236.
Wolf, M. J., and Gross, R. W. (1996) J Biol Chem 271, 20989-20992.
Jenkins, C. M., Wolf, M. J., Mancuso, D. J., and Gross, R. W. (2001) J Biol Chem 276, 7129-7135.
Bolotina, V. M. (2004) Sci STKE 2004, pe34.
Randriamampita, C., and Tsien, R. Y. (1993) Nature 364, 809-814.
Randriamampita, C., and Tsien, R. Y. (1995) J Biol Chem 270, 29-32.
Hazen, S. L., and Gross, R. W. (1991) J Biol Chem 266, 14526-14534.
Hazen, S. L., and Gross, R. W. (1991) Biochem J 280 (Pt 3), 581-587.
Jenkins, C. M., Han, X., Mancuso, D. J., and Gross, R. W. (2002) J Biol Chem 277, 32807-32814.
Cohen Simonsen, A., Bernchou Jensen, U., Faergeman, N. J., Knudsen, J., and Mouritsen, O. G. (2003) FEBS Lett 552, 253-258.
Chao, H., Martin, G. G., Russell, W. K., Waghela, S. D., Russell, D. H., Schroeder, F., and Kier, A. B. (2002) Biochemistry 41, 10540-10553.
Hazen, S. L., Stuppy, R. J., and Gross, R. W. (1990) J Biol Chem 265, 10622-10630.
Wessel, D., and Flugge, U. I. (1984) Anal Biochem 138, 141-143.
Laemmli, U. K. (1970) Nature 227, 680-685.
Nesterenko, M. V., Tilley, M., and Upton, S. J. (1994) J Biochem Biophys Methods 28, 239-242.
Gross, R. W. (1984) Biochim Biophys Acta 802, 197-202.
Hunt, M. C., Solaas, K., Kase, B. F., and Alexson, S. E. (2002) J Biol Chem 277, 1128-1138.
Hazen, S. L., Zupan, L. A., Weiss, R. H., Getman, D. P., and Gross, R. W. (1991) J Biol Chem 266, 7227-7232.
Zupan, L. A., Weiss, R. H., Hazen, S. L., Parnas, B. L., Aston, K. W., Lennon, P. J., Getman, D. P., and Gross, R. W. (1993) J Med Chem 36, 95-100.
O'Brien, P. J., St Jules, R. S., Reddy, T. S., Bazan, N. G., and Zatz, M. (1987) J Biol Chem 262, 5210-5215.
Duncan, J. A., and Gilman, A. C. (1996) J Biol Chem 271, 23594-23600.
Tu, Y., Wang, J., and Ross, E. M. (1997) Science 278, 1132-1135.
Ford, D. A., Homer, C. C., and Gross, R. W. (1998) Biochemistry 37, 11953-11961.
Schaller, J. (2000) Methods Mol Biol 146, 425-437.
Quach, T. T., Li, N., Richards, D. P., Zheng, J., Keller, B. O., and Li, L. (2003) J Proteome Res 2, 543-5 52.
Zischka, H., Gloeckner, C. J., Klein, C., Willmann, S., Swiatek-de Lange, M., and Ueffing, M. (2004) Proteomics 4, 3776-3782.
Eichacker, L. A., Granvogl, B., Mirus, O., Muller, B. C., Miess, C., and Schleiff, E. (2004) J Biol Chem 279, 50915-50922.
Nowatzke, W., Ramanadham, S., Ma, Z., Hsu, F. F., Bohrer, A., and Turk, J. (1998) Endocrinology 139, 4073-4085.
Thomas, D., and Hanley, M. R. (1995) J Biol Chem 270, 6429-6432.
Han, X., and Gross, R. W. (1994) Proc Natl Acad Sci USA 91, 10635-10639.
Wolf, R. A., and Gross, R. W. (1985) J Biol Chem 260, 7295-7303.
McHowat, J., and Creer, M. H. (1998) Am J Physiol 274, C447-454.
Murakami, M., Shimbara, S., Kambe, T., Kuwata, H., Winstead, M. V., Tischfield, J. A., and Kudo, I. (1998) J Biol Chem 273, 14411-14423.
Tessier, C., Hichami, A., and Khan, N. A. (2002) FEBS Lett 520, 111-116.
Ma, Z., Ramanadham, S., Wohltmann, M., Bohrer, A., Hsu, F. F., and Turk, J. (2001) J Biol Chem 276, 13198-13208.
Hazen, S. L., Ford, D. A., and Gross, R. W. (1991) J Biol Chem 266, 5629-5633.
Williams, S. D., and Gottlieb, R. A. (2002) Biochem J 362, 23-32.
Ma, Z., Ramanadham, S., Kempe, K., Chi, X. S., Ladenson, J., and Turk, J. (1997) J Biol Chem 272, 11118-11127.
Larsson Forsell, P. K., Kennedy, B. P., and Claesson, H. E. (1999) Eur J Biochem 262, 575-585.
Larsson, P. K., Claesson, H. E., and Kennedy, B. P. (1998) J Biol Chem 273, 207-214.
Atsumi, U., Murakami, M., Kojima, K., Hadano, A., Tajima, M., and Kudo, I. (2000) J Biol Chem 275, 18248-18258.
Lauber, K., Bohn, E., Krober, S. M., Xiao, Y. J., Blumenthal, S. G., Lindemann, R. K., Marini, P., Wiedig, C., Zobywalski, A., Baksha, S., Xu, Y., Autenrieth, I. B., Schulze-Osthoff, K., Belka, C., Stuhler, G., and Wesselborg, S. (2003) Cell 113, 717-730.
Tay, H. K., and Melendez, A. J. (2004) J Biol Chem 279, 22505-22513.
Wang, Z., Ranianadham, S., Ma, Z. A., Bao, S., Mancuso, D. J., Gross, R. W., and Turk, J. (2005) J Biol Chem 280, 6840-6849.
Jenkins, C. M., Han, X., Yang, J., Mancuso, D. J., Sims, H. F., Muslin, A. J., and Gross, R. W. (2003) Biochemistry 42, 11798-11807.
Lehner, R., and Kuksis, A. (1993) J Biol Chem 268, 24726-24733.
Liu, G. X., Hanley, P. J., Ray, J., and Daut, J. (2001) Circ Res 88, 918-924.
Rohacs, T., Lopes, C. M., Jin, T., Ramdya, P. P., Molnar, Z., and Logothetis, D. E. (2003) Proc Natl Acad Sci USA 100, 745-750.
Mignen, O., Thompson, J. L., and Shuttleworth, T. J. (2003) J Biol Chem 278, 10174-10181.
Gubitosi-Klng, R. A., Yu, S. P., Choi, D. W., and Gross, R. W. (1995) J Biol Chem 270, 2885-2888.
Honore, E., Barhanin, J., Attali, B., Lesage, F., and Lazdunski, M. (1994) Proc Natl Acad Sci USA 91, 1937-1941.
Hamilton, K. L., Syme, C. A., and Devor, D. C. (2003) J Biol Chem 278, 16690-16697.
Carattino, M. D., Hill, W. G., and Kleyman, T. R. (2003) J Biol Chem 278, 36202-36213.
Paucek, P., Yarov-Yarovoy, V., Sun, X., and Garlid, K. D. (1996) J Biol Chem 271, 32084-32088.
Bolotina, V. M., and Csutora, P. (2005) Trends Biochem Sci 30, 378-3 87.
Kim, Y. C., Gomez, F. E., Fox, B. G., and Ntambi, J. M. (2000) J Lipid Res 41, 1310-1316.
Resh, M. D. (1999) Biochim Biophys Acta 1451, 1-16.
Bijlmakers, M. J., and Marsh, M. (2003) Trends Cell Biol 13, 32-42.
Smotrys, J. E., and Linder, M. E. (2004) Annu Rev Biochem 73, 559-587.

What is claimed is:

1. A method of treating a living mammal having diabetic cardiomyopathy comprises administering an effective amount of an inhibitor to the mammal performing a shotgun lipidomics analysis on the mammal and determining that the treatment was successful when and if serum or tissue biopsy cardiolipin levels are increased and/or lysocardiolipin levels are decreased.

2. A method in accordance with claim 1 wherein the shotgun lipid analysis shows that cardiolipin molecular species are increased and lysocardiolipin molecular species are decreased in a diabetic mammal as compared to a shotgun lipid analysis performed on a nonafflicted individual displaying normal cardiolipin levels in a normal mammal.

3. A method in accordance with claim 1 wherein the subject is a living human.

4. A method in accordance with claim 1 wherein performing a shotgun lipidomics analysis comprises isolating at least one of serum and a tissue sample, extracting total lipids into chloroform/methanol, and analyzing and quantifying cardiolipin/lysocardiolipin molecular species by ESI/MS.

5. A method in accordance with claim 1 wherein the cardiomyopathy presented is capable of being reversed.

6. A method in accordance with claim 1 wherein administration of an inhibitor of iPLA₂β and/or iPLA₂γ results in increased cardiolipin levels resulting in reversal of the cardiomyopathy.

7. A method of treating a living mammal afflicted with cardiomyopathy comprises administering to the mammal an effective amount of a gene whose express increases the synthesis of cardiolipin.

8. A method in accordance with claim 7 wherein the gene encodes a protein.

9. A method in accordance with claim 7 wherein the gene comprises a polynucleotide having a SEQUENCE NO. 1.

10. A method in accordance with claim 7 wherein the gene comprises a polynucleotide having a SEQUENCE NO. 3.

11. A method in accordance with claim 7 wherein the encoded and expressed protein comprises a polypeptide having a SEQUENCE NO. 2.

12. A method in accordance with claim 7 wherein the encoded and expressed protein comprises a polypeptide having a SEQUENCE NO. 4.

13. A method in accordance with claim 7 wherein the subject is a living mouse.

14. A method in accordance with claim 7 wherein the subject is a living human.

15. A method in accordance with claim 7 wherein administration of an inhibitor of iPLA₂β and/or iPLA₂γ results in increased cardiolipin levels resulting in reversal of the cardiomyopathy.

16. A method to detect diabetic cardiomyopathy in a living mammal, the method comprising analyzing a representative portion of the mammal by using shotgun lipodomics thereon to obtain and determine the cardiolipin content, comparing the obtained cardiolipin content with a cardiolipin content range previously obtained and associated for a corresponding non-diabetic mammal and determining that the analyzed mammal has diabetic cardiomyopathy when the analyzed cardiolipin content is about 5% to about 95% less than the cardiolipin content usually expected in the cells of a non-afflicted mammal.

17. A method in accordance with claim 16 wherein determining that the analyzed mammal has diabetic cardiomyopathy comprises determining that the analyzed mammal has diabetic cardiomyopathy when the analyzed cardiolipin content is about 25% to about 75% less than the cardiolipin content usually expected in the cells of a non-afflicted mammal.


SEQUENCE LISTING (5 sequences)

SEQ ID NO: 1 - DNA, 906 bp, Homo sapiens
atgctagccttgcgcgtggcgcgcggctcgtggggggccctgcgcggcgccgcttgggctccgggaacgcggccgagtaagcgacgcgcctgctgggccctgctgccgcccgtgccctgctgcttgggctgcctggccgaacgctggaggctgcgtccggccgctcttggcttgcggctgcccgggatcggccagcggaaccactgttcgggcgcggggaaggcggctcccaggccagcggccggagcgggcgccgctgccgaagccccgggcggccagtggggcccggcgagcacccccagcctgtatgaaaacccatggacaatcccgaatatgttgtcaatgacgagaattggcttggccccagttctgggctatttgattattgaagaagattttaatattgcactaggagtttttgctttagctggactaacagatttgttggatggatttattgctcgaaactgggccaatcaaagatcagctttgggaagtgctcttgatccacttgctgataaaatacttatcagtatcttatatgttagcttgacctatgcagatcttattccagttccacttacttacatgatcatttcgagagatgtaatgttgattgctgctgttttttatgtcagataccgaactcttccaacaccacgaacacttgccaagtatttcaatccttgctatgccactgctaggttaaaaccaacattcatcagcaaggtgaatacagcagtccagttaatcttggtggcagcttctttggcagctccagttttcaactatgctgacagcatttatcttcagatactatggtgttttacagctttcaccacagctgcatcagcttatagttactatcattatggccggaagactgttcaggtgataaaagactga

SEQ ID NO: 2 - protein, 301 aa, Homo sapiens
MLALRVARGSWGALRGAAWAPGTRPSKRRACWALLPPVPCCLGCLAERWRLRPAALGLRLPGIGQRNHCSGAGKAAPRPAAGAGAAAEAPGGQWGPASTPSLYENPWTIPNMLSMTRIGLAPVLGYLIIEEDFNIALGVFALAGLTDLLDGFIARNWANQRSALGSALDPLADKILISILYVSLTYADLIPVPLTYMIISRDVMLIAAVFYVRYRTLPTPRTLAKYFNPCYATARLKPTFISKVNTAVQLILVAASLAAPVFNYADSIYLQILWCFTAFTTAASAYSYYHYGRKTVQVIKD

SEQ ID NO: 3 - DNA, 618 bp, Homo sapiens
atgctagccttgcgcgtggcgcgcggctcgtggggggccctgcgcggcgccgcttgggctccgggaacgcggccgagtaagcgacgcgcctgctgggccctgctgccgcccgtgccctgctgcttgggctgcctggccgaacgctggaggctgcgtccggccgctcttggcttgcggctgcccgggatcggccagcggaaccactgttcgggcgcggggaaggcggctcccaggccagcggccggagcgggcgccgctgccgaagccccgggcggccagtggggcccggcgagcacccccagcctgtatgaaaacccatggacaatcccgaatatgttgtcaatgacgagaattggcttggccccagttctgggctatttgattattgaagaagattttaatattgcactaggagtttttgctttagctggactaacagatttgttggatggatttattgctcgaaactgggccaatcaaagatcagctttgggaagtgctcttgatccacttgctgataaaatacttatcagtatcttatatgttagcttgacctatgcagatcttattccagcgaacacttgccaagtatttcaatccttgctatgccactgctag

SEQ ID NO: 4 - protein, 205 aa, Homo sapiens
MLALRVARGSWGALRGAAWAPGTRPSKRRACWALLPPVPCCLGCLAERWRLRPAALGLRLPGIGQRNHCSGAGKAAPRPAAGAGAAAEAPGGQWGPASTPSLYENPWTIPNMLSMTRIGLAPVLGYLIIEEDFNIALGVFALAGLTDLLDGFIARNWANQRSALGSALDPLADKILISILYVSLTYADLIPANTCQVFQSLLCHC

SEQ ID NO: 5 - protein, 752 aa, Homo sapiens [sequence omitted]